(19) United States
(12) Patent Application Publication
(10) Pub. No.: US 2004/0087014 A1
(43) Pub. Date: May 6, 2004

(54) Eukaryotic expression libraries and methods of use

(76) Inventor: William D Huse, Del Mar, CA (US)

Correspondence Address: Cathryn Campbell, McDermott Will & Emery, Suite 700, 4370 La Jolla Village Drive, San Diego, CA, 92122, US

(21) Appl. No.: 10/433,206

(22) PCT Filed: Nov. 28, 2001
(86) PCT No.: PCT/US01/44600

(30) Foreign Application Priority Data
Nov. 28, 2000 (US) 09/724,762

(51) Int. Cl. C12N 5/02

(52) U.S. Cl. 435/325

(57) ABSTRACT

The invention provides a cell composition comprising a population of non-yeast eukaryotic cells containing a diverse population of variant nucleic acids, each of the variant nucleic acids being expressed in a different cell and located within each cell at an identical site in the genome. The invention also provides a method of identifying a polypeptide exhibiting optimized activity by screening a population of non-yeast eukaryotic cells containing a diverse population of variant nucleic acids for an activity associated with a parent polypeptide of a diverse population of variant polypeptides encoded by the variant nucleic acids; and identifying a variant polypeptide exhibiting an optimized activity relative to the parent polypeptide.

This invention was made with government support under grant number NIH 1 R43 GM60106-01 awarded by the National Institutes of Health. The United States Government has certain rights in this invention.

### BACKGROUND OF THE INVENTION

The present invention relates generally to molecular biology and more specifically to eukaryotic expression libraries.

The development of new and more effective drugs is a primary goal of the pharmaceutical industry. Drug discovery and development can be described as following two general approaches, screening for lead compounds and structure-based drug design.

Drug discovery based on screening for lead compounds involves generating a pool of candidate compounds. These candidate compounds can be derived from natural products, such as plants, insects or other organisms. The pool of candidate compounds can also be recombinantly generated such as with phage display libraries of combinatorial antibody libraries and random peptide libraries. Alternatively, the candidate compounds can be chemically synthesized using approaches such as combinatorial chemistry in which compounds are synthesized by combining chemical groups to generate a large number of diverse candidate compounds.

Generally, the pool of candidate compounds is screened with a drug target of interest to identify potential lead compounds. This approach usually requires assaying large numbers of compounds for a desired activity. Depending on the assay, compound availability and preparation, the screening of a pool of candidate compounds can be laborious and time consuming. Moreover, further rounds of manipulations such as the screening of modified forms of the lead compound are additionally performed to determine a structure with optimal activity. Thus, these additional manipulations further complicate and increase the time and labor required for the development of a drug candidate which exhibits optimal binding activity to the target of interest.

Drug discovery and development relying on structure-based drug design uses a three-dimensional structure prediction of the drug target as a template to model compounds which inhibit or otherwise interfere with critical residues that are required for activity in the target molecule. Model compounds which show activity toward the drug target are then used as lead compounds for the development of candidate drugs which exhibit a desired activity toward the drug target.

Identifying model compounds using structure-based drug design can provide advantages in predicting modifications of the lead compound that will likely improve binding of the compound to the drug target. However, obtaining structures of relevant drug targets is extremely time consuming and laborious. Moreover, successive rounds of modifications and testing to identify a compound which exhibits a desired binding activity toward the drug target is similarly laborious and time consuming. Such a process often takes years to accomplish. In addition, if the drug target of interest is a receptor on the surface of cells, it can be embedded in the cell membrane. Determination of the three-dimensional structures of such membrane proteins is extremely difficult as evidenced by the limited number of membrane protein structures currently available.

Another difficulty in identifying drug candidates based on structure-function studies of a target is characterizing the drug candidate and target interactions in a system that more accurately reflects the physiological environment in which the interaction would occur. Due to the convenience and inexpensive nature of bacterial expression systems, many initial structure-function studies of eukaryotic proteins are conducted using bacterial expression systems and bacterial expression libraries. However, such bacterial expression systems are unable to incorporate many of the post-translational modifications that normally occur in eukaryotic cells. Furthermore, bacterial systems often result in expression of insoluble forms of eukaryotic proteins, thus limiting the ability to obtain meaningful information on drug candidate interactions.

Although expression of eukaryotic proteins in eukaryotic cells would allow post-translational modification and circumvent solubility problems due to bacterial expression, eukaryotic expression systems also have limitations. For example, the expression of combinatorial protein libraries in mammalian cells has been hampered by limitations associated with the transformation of mammalian cells. DNA-mediated transformation of mammalian cells typically results in the random integration of exogenous DNA into the host genome, leading to significant variability in protein expression. In addition, experimental conditions that ensure transformation efficiencies necessary and sufficient for the expression of protein libraries can lead to integration of the DNA at multiple sites in each cell (Lacy et al., Cell, 34:343-358 (1983)). Consequently, a single cell may express multiple distinct protein variants, significantly complicating both screening and subsequent identification of the mutation by DNA sequencing.

Homologous recombination has been used to target a single copy of DNA to a specific location in the genome. However, complexities associated with the methodology and a large number of spurious targeting events has hampered the use of homologous recombination for the efficient expression of combinatorial protein libraries (Lin et al., Proc. Natl. Acad. Sci. USA, 82:1391-1395 (1985); Thomas et al., Cell, 44:419-428 (1986)).

Thus, there exists a need for eukaryotic expression systems useful for expressing and screening libraries for structure-function studies and drug discovery. The present invention satisfies this need and provides related advantages as well.

### SUMMARY OF THE INVENTION

The invention provides a cell composition comprising a population of non-yeast eukaryotic cells containing a diverse population of variant nucleic acids, each of the variant nucleic acids being expressed in a different cell and located within each cell at an identical site in the genome. The invention also provides a method of identifying a polypeptide exhibiting optimized activity by screening a population of non-yeast eukaryotic cells containing a diverse population of variant nucleic acids for an activity associated with a parent polypeptide of a diverse population of variant polypeptides encoded by the variant nucleic acids; and identifying a variant polypeptide exhibiting an optimized activity relative to the parent polypeptide.

### BRIEF DESCRIPTION OF THE DRAWINGS

FIG. 1 shows binding of chemical ligand, represented as a point in space designated X, to a receptor, represented as a disc. The bottom panel shows distribution of ligands where open circles represent diverse ligands and closed circles represent focused ligands.

FIG. 2 shows identification of an optimal binding ligand using a receptor represented as three discs and a ligand represented as three points designated X.

FIGS. 3A-3D show binding of anti-idiotypic antibody ligands to BR96 antibody receptor variants.

FIG. 4 shows identification of an optimal binding anti-idiotypic antibody ligand that binds to multiple antibody receptor variants.

FIG. 5 shows the components of the doublelox strategy. FIG. 5A shows the recombinase recognition sequence (underlined) and cleavage sites (arrows) for loxP (SEQ ID NO:29). FIG. 5B shows the recombinase recognition sequence (underlined) and cleavage sites (arrows) for lox511 (SEQ ID NO:30). The “*” denotes the change in lox511 from loxP. FIG. 5C shows the steps of Cre-mediated double crossover.

FIG. 6 shows a comparison of the amino acid sequence of Sh ble gene product (SEQ ID NO:31) with related proteins encoded by the different genes Sa ble (SEQ ID NO:32) and Tn5 ble (SEQ ID NO:33) (Gatignol et al., FEBS Lett. 230:171-175 (1988)). Residues of the Sh ble gene product (BRP) putatively involved in bleomycin binding are indicated with an asterisk while conserved residues are shaded.

FIG. 7 shows Zeocin screening of BRP libraries expressed in 13-1 mammalian cells. Cell proliferation is indicated by (+), while toxicity is indicated by (−).

FIG. 8 shows the amino acid sequence of human butyrylcholinesterase (SEQ ID NO:89) with seven regions used to generate focused libraries underlined. The aromatic active gorge residues are W82, W112, Y128, W231, F329, Y332, W430 and Y440.

### DETAILED DESCRIPTION OF THE INVENTION

The invention provides compositions comprising a population of non-yeast eukaryotic cells containing a diverse population of variant nucleic acids or heterologous nucleic acids and methods of using the populations. The compositions comprise a population of non-yeast eukaryotic cells containing a diverse population of variant nucleic acids or heterologous nucleic acids, each species of nucleic acid being expressed in a different cell and located within each cell at an identical site in the genome. The compositions and methods are advantageous in that each nucleic acid in a population of nucleic acids can be expressed in a separate-cell to minimize complications associated with transfection of multiple species in the same cell. The nucleic acids can also be targeted to the same site in the cell genome, for example, using site-specific recombination, to generate isogenic cells expressing the nucleic acids.

The invention population of cells containing variant nucleic acids or heterologous nucleic acid fragments are useful in allowing convenient characterization and comparison of polypeptides encoded by the nucleic acids without the variability due to random integration or copy number effects of transfected nucleic acids. The methods of the invention are applicable to directed evolution in which characteristics of a molecule are optimized by generating and screening variant molecules for a preferred activity.

Rapid and efficient methods for determining optimal ligand-receptor binding partners are disclosed herein. The methods are applicable for the identification of specific ligands to desired target molecules. Such ligands can be developed as potential drug candidates or, alternatively, used as lead compounds for the generation and identification of ligand variants which exhibit enhanced activity of the desired binding property. The methods are advantageous in that they use a population of receptor variants to rapidly identify ligands that have a high likelihood of binding to the target receptor molecule. By initially screening with a population of variants to the target receptor, the probability of detecting binding events is increased obtaining increased binding events is productive because the use of receptor variants that are all related to a parent receptor results in the identification of binding events similar to the parent receptor and, therefore, ligands identified by such a screen are similarly related to those ligands that will associate with and bind to the parent receptor. Therefore, the initial screen using a population of variants results in the rapid identification and enrichment for ligands having favorable binding characteristics toward the target receptor. This enriched population can then be subsequently screened for ligands having optimal binding characteristics toward the target receptor. The methods of the invention therefore provide a rapid and efficient method for the identification of specific ligands which are applicable for the diagnosis and treatment of diseases.

As used herein, the term “receptor” is intended to refer to a molecule of sufficient size so as to be capable of selectively binding a ligand. Such molecules generally are macromolecules, such as polypeptides, nucleic acids, carbohydrate or lipid. However, derivatives, analogues and mimetic compounds as well as natural or synthetic organic compounds are also intended to be included within the definition of this term. The size of a receptor is not important so long as the receptor exhibits or can be made to exhibit selective binding activity to a ligand. Furthermore, the receptor can be a fragment or modified form of the entire molecule so long as it exhibits selective binding to a desired ligand. For example, if the receptor is a polypeptide, a fragment or domain of the native polypeptide which maintains substantially the same binding selectivity as the intact polypeptide is intended to be included within the definition of the term receptor. Specific examples of such a binding domain or fragment is the variable region of an antibody molecule. Complementarity determining regions (CDR) within the variable region can also exhibit substantially the same binding selectivity as the antibody molecule and are therefore considered to be within the meaning of the term.

An optimal binding ligand is identified by generating a population of receptor variants. The receptor variants can be pooled into a collective receptor variant population for screening or the receptor variants can be screened individually for binding activity to ligands. The receptor variant population can be screened by dividing the ligand population into subpopulations or individual ligands to determine binding activity. The binding activity of ligands exhibiting binding to the receptor variant population are compared to identify a ligand having optimal binding characteristics. Further optimization of binding ligands can be performed. After identifying a ligand having optimal binding characteristics, further optimized binding ligands can be subsequently identified by generating a library of ligand variants based on the identified optimal binding ligand and screening for binding activity to the parent receptor. The binding activity of positive binding ligand variants are compared to each other and to the parent ligand to identify the ligand or ligands which exhibit preferred or optimal binding characteristics to the parent receptor.

Receptors can include, for example, cell surface receptors such as G protein coupled receptors, integrins, growth factor receptors and cytokine receptors. In one embodiment, an optimal binding ligand is identified by generating a population of G protein coupled receptor variants. The G protein coupled receptor variants are pooled into a collective receptor variant population and screened for binding activity to ligands within a diverse population. Receptors can also be antibodies and can include other polypeptides or ligands of the immune system. Such other polypeptides of the immune system include, for example, T cell receptors (TCR), major histocompatibility complex (MHC), CD4 receptor and CD8 receptor. Furthermore, cytoplasmic receptors such as steroid hormone receptors and DNA binding polypeptides such as transcription factors and DNA replication factors are likewise included within the definition of the term receptor. Another exemplary receptor is the bleomycin resistance protein (BRP), which confers resistance to bleomycin (see Examples VII, IX and X). An additional exemplary receptor is butyrylcholinesterase, which hydrolyzes choline esters (see Example XI).

As used herein, the term “polypeptide” when used in reference to a receptor or a ligand is intended to refer to peptide, polypeptide or protein of two or more amino acids. The term is similarly intended to refer to derivatives, analogues and functional mimetics thereof. For example, derivatives can include chemical modifications of the polypeptide such as alkylation, acylation, carbamylation, iodination, or any modification which derivatizes the polypeptide. Analogues can include modified amino acids, for example, hydroxyproline or carboxyglutamate, and can include amino acids that are not linked by peptide bonds. Mimetics encompass chemicals containing chemical moieties that mimic the function of the polypeptide regardless of the predicted three-dimensional structure of the compound. For example, if a polypeptide contains two charged chemical moieties in a functional domain, a mimetic places two charged chemical moieties in a spatial orientation and constrained structure so that the charged chemical function is maintained in three-dimensional space. Thus, all of these modifications are included within the term “polypeptide” so long as the polypeptide retains its binding function.

As used herein, the term “ligand” refers to a molecule that can selectively bind to a receptor. The term selectively means that the binding interaction is detectable over non-specific interactions by a quantifiable assay. A ligand can be essentially any type of molecule such as polypeptide, nucleic acid, carbohydrate, lipid, or small organic compound. Moreover, derivatives, analogues and mimetic compounds are also intended to be included within the definition of this term. As such, a molecule that is a ligand can also be a receptor and, conversely, a molecule that is a receptor can also be a ligand since ligands and receptors are defined as binding partners. Those skilled in the art know what is intended by the meaning of the term ligand. Specific examples of ligands are natural or synthetic organic compounds as well as recombinantly or synthetically produced polypeptides. Such polypeptides that bind to receptor variants are described below in Example V.

As used herein, the term “variant” when used in reference to a receptor or ligand is intended to refer to a molecule that shares a similar structure and function but differs by at least a single atom from a parent molecule. The characteristics that define the function can be determined by a parent receptor or by a parent ligand. Variants possess, for example, substantially the same or similar binding function as the parent molecule. However, variants can have a detectable difference in the chemical functional groups of the binding function and still be considered a variant of the parent molecule so long as the binding function is similar. Variants include, for example, parent receptors that are directly modified such as by the mutation of an amino acid residue or the addition of a chemical moiety. Modifications can also be indirect such as the binding of a regulatory molecule or allosteric effector which alters the binding function of the parent receptor.

Additionally, the variant can be an isoform or family member that is distinct but related to the parent receptor. All of such direct or indirect modifications of a parent molecule as well as related members thereof are considered to be within the definition of the term variant as used herein. Chemical functional groups that differ from the parent molecule can be used to generate a population of variant molecules. In the specific example of a polypeptide receptor parent, a variant can differ by, for example, one or more amino acids in a functional binding domain. In this specific example, a functional binding domain refers to a region or a portion of the polypeptide that contributes to binding interactions between the receptor and ligand. Such functional binding domains include, for example, both catalytic domains and ligand binding domains, as well as structural domains that contribute to the polypeptide function.

As used herein, the term “population” is intended to refer to a group of two or more different molecules. A population can be as large as the number of individual molecules currently available to the user or able to be made by one skilled in the art. Typically, populations can be as small as 2 molecules and as large as 10¹³ molecules. In some embodiments, populations are between about 5 and 10 different species as well as up to hundreds or thousands of different species. In the specific example presented in Example V, the population described therein is 7 different species. Example IX exemplifies populations of about 200 to about 1300 different species. In other embodiments, populations can be, for example, greater than 10⁵, 10⁶ and 10⁸ different species. In yet other embodiments, populations are between about 10⁸-10¹² or more different species. The 5 populations of the invention can therefore be about 10 or more, about 15 or more, about 20 or more, about 30 or more, about 40 or more, about 50 or more, about 75 or more, about 100 or more, about 150 or more, about 200 or more, about 250 or more, about 300 or more, about 350 or more, about 400 or more, about 450 or more, about 500 or more, about 700 or more, about 800 or more, about 1000 or more, about 2000 or more, about 5000 or more, about 1×10⁴ or more, about 1×10⁵ or more, about 1×10⁶ or more, about 1×10⁷ or more, or even about ×10⁸ or more different species. Moreover, the populations can be diverse or redundant depending on the intent and needs of the user. Those skilled in the art will know what size and diversity of a population is suitable for a particular application.

As used herein, the term “subpopulation” refers to a subgroup of one or more species of molecules from an original population. The subpopulation can be obtained by, for example, dividing the population into one or more fractions or synthesizing or generating a known fraction of the original population. The subpopulation need not contain equivalent numbers of different molecules.

As used herein, the term “collective,” when used in reference to populations or subpopulations, refers to an aggregate or pool of members that form the population or subpopulation such that members of the population can intermingle. In contrast, a non-collective population is one in which individual members of the population are segregated rather than aggregated, for example, segregated into individual wells of a plate.

As used herein, the term “optimal binding” refers to a preferred binding characteristic of a ligand and receptor interaction. Optimal binding can be ligand-receptor interactions of a desired affinity, avidity or specificity. For example, optimal binding can be interactions that are most effective in a biological assay. The optimal binding characteristics will depend on the particular application of the binding molecule. For example, the binding standard can be relative affinity of a ligand for the parent receptor. In this case, a ligand in a population with the highest binding affinity to a parent receptor would have optimal binding. Alternatively, the standard can be the highest binding affinity of a ligand subpopulation to a receptor variant subpopulation. In this example, the ligand subpopulation with highest affinity for a receptor variant subpopulation would have optimal binding. In this case, the highest affinity ligand would be a member of the ligand subpopulation and, likewise, the highest affinity receptor variant would be a member of the receptor variant subpopulation optimal binding also can be binding to the largest number of receptor variants or binding to greater than some threshold number of receptor variants. In some applications, lower affinity binding can be optimal binding.

As used herein, the term “heterologous nucleic acid” refers to a nucleic acid that is not naturally expressed in a particular cell.

The invention provides a cell composition comprising a population of non-yeast eukaryotic cells containing a diverse population of about 10 or more variant nucleic acids, each of the variant nucleic acids being expressed in a different cell and located within each cell at an identical site in the genome. If desired, the cell compositions can contain variant nucleic acids having predetermined amino acid changes at preselected positions within a parent amino acid sequence.

The incorporation of variant nucleic acids or heterologous nucleic acid fragments at an identical site in the genome functions to create isogenic cell lines that differ only in the expression of a particular variant or heterologous nucleic acid. Incorporation at a single site minimizes positional effects from integration at multiple sites in a genome that affect transcription of the mRNA encoded by the nucleic acid and complications from the incorporation of multiple copies or expression of more than one nucleic acid species per cell.

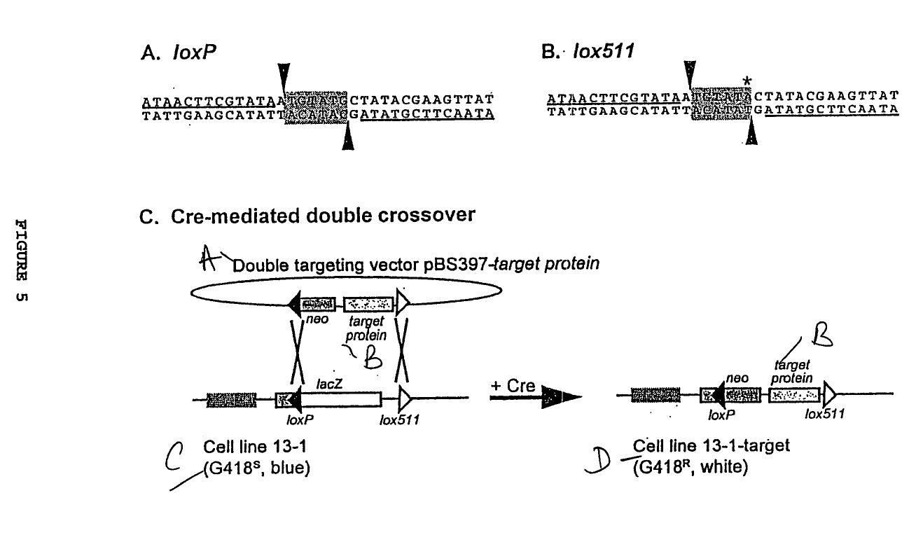

One approach for targeting variant or heterologous nucleic acids to a single site in the genome uses Cre recombinase to target insertion of exogenous DNA into the eukaryotic genome at a site containing a site specific recombination sequence (Sauer and Henderson, Proc. Natl. Acad. Sci. USA, 85:5166-5170 (1988); Fukushige and Sauer, Proc. Natl. Acad. Sci. U.S.A. 89:7905-7909 (1992); Bethke and Sauer, Nuc. Acids Res., 25:2828-2834 (1997)). Cre recombinase is a well-characterized 38-kDa DNA recombinase (Abremski et al., Cell 32:1301-1311 (1983)) that is both necessary and sufficient for sequence-specific recombination in bacteriophage P1. Recombination occurs between two 34-base pair loxP sequences each consisting of two inverted 13-base pair recombinase recognition sequences (FIG. 5A, underlined) that surround a core region (FIG. 5A, shaded box) (Sternberg and Hamilton, J. Mol. Biol. 150:467-486 (1981a); Sternberg and Hamilton, J. Mol. Biol., 150:487-507 (1981b) DNA cleavage and strand exchange occurs on the top or bottom strand at the edges of the core region (FIG. 5A, arrows). Cre recombinase also catalyzes site-specific recombination in eukaryotes, including both yeast (Sauer, Mol. Cell. Biol. 7:2087-2096 (1987)) and mammalian cells (Sauer and Henderson, Proc. Natl. Acad. Sci. USA, 85:5166-5170 (1988); Fukushige and Sauer, Proc. Natl. Acad. Sci. U.S.A. 89:7905-7909 (1992); Bethke and Sauer, Nuc. Acids Res., 25:2828-2834 (1997)).

In addition to Cre recombinase, Flp recombinase can also be used to target insertion of exogenous DNA into a particular site in the genome (O'Gorman et al., Science 251:1351-1355 (1991); Dymecki, Proc. Natl. Acad. Sci. U.S.A. 93:6191-6196 (1996)). The target site for Flp recombinase consists of 13 base-pair repeats separated by an 8 base-pair spacer: 5-GAAGTTCCTATTC(TCTAGAAA)GTATAGGAACTTC-3′ (SEQ ID NO:90). It is understood that any combination of site-specific recombinase and corresponding recombination site can be used in methods of the invention to target a nucleic acid to a particular site in the genome.

The recombinase can be encoded on a vector that is co-transfected with a vector containing variant nucleic acids or heterologous nucleic acid fragments. Alternatively, the expression element encoding a recombinase can be incorporated into the same vector expressing the nucleic acid variants or heterologous nucleic acid fragments. In addition to simultaneously transfecting the nucleic acid encoding a recombinase with the nucleic acids encoding variant nucleic acids or heterologous nucleic acid fragments, a vector encoding the recombinase can be transfected into a cell, and the cells can be selected for expression of recombinase. A cell stably expressing the recombinase can subsequently be transfected with nucleic acids encoding variant nucleic acids or heterologous nucleic acid fragments.

As exemplified herein, the precise site-specific DNA recombination mediated by Cre recombinase has been used to create stable mammalian transformants containing a single copy of exogenous DNA (see Example VII). The frequency of Cre-mediated targeting events was also enhanced substantially using a modified doublelox strategy. The doublelox strategy is based on the observation that certain nucleotide changes within the core region of the lox site (FIG. 5B, asterisk) alter the site selection specificity of Cre-mediated recombination with little effect on the efficiency of recombination (Hoess et al., Nucleic Acids Res. 14:2287-2300 (1986)). Thus, incorporation of loxP and an altered loxP site, termed lox511 (FIG. 5B), in both the targeting vector and the host cell genome results in site-specific recombination by a double crossover event (FIG. 5C). The doublelox approach increases the recovery of site-specific integrants by 20-fold over the single crossover insertional recombination, increasing the absolute frequency of site-specific recombination such that it exceeds the frequency of illegitimate recombination (Bethke and Sauer, Nuc. Acids Res., 25:2828-2834 (1997)). Indeed, the frequency of targeted integration was 1% of the total number of viable mammalian cells plated with an estimated transfection efficiency of 16% (Bethke and Sauer, Nuc. Acids Res., 25:2828-2834 (1997)).

Homologous recombination can also be used to locate a nucleic acid sequence at a particular site in the genome. For example, a vector can be designed so that an individual nucleic acid of a population of nucleic acids is flanked by nucleic acid sequences having sufficient homology to allow homologous recombination with a homologous nucleic acid sequence located at a particular site in the genome of a cell. Such a homologous sequence can naturally occur at a particular genomic location or the homologous sequence can be introduced recombinantly using well known methods of transfection and using vectors that allow integration into the host genome. If the homologous sequence is introduced into the genome recombinantly, a cell line can be clonally isolated so that cells of a given clone will have the homologous sequence located at the same genomic site. Methods of introducing a nucleic acid into the genome at a particular site using homologous recombination use the endogenous recombination machinery rather than an exogenous recombinase such as Cre of Flp.

The region of homology flanking an invention nucleic acid is sufficient to allow homologous recombination with the homologous sequence located at a particular site in the genome. Such homologous sequences will generally have a length of at least about 1 kb, more preferably about 2 kb. Generally, the rate of homologous recombination increases with increasing length of homologous DNA sequence, up to limits that are estimated at up to 15 kb (see Ausubel et al., Current Protocols in Molecular Biology, John Wiley & Sons, New York (1999)).

It is understood that the degree of homology between the construct and target genome can have an effect on the rate of homologous recombination. Homologous recombination requires stretches of exact DNA homology such that a single DNA mismatch is sufficient to reduce the rate of homologous recombination (Deng and Capecchi, Mol. Cell. Biol. 12:3365-3371(1992)). Thus, a region of homology flanking an invention nucleic acid that is sufficient to allow homologous recombination with the homologous sequence located at a particular site in the genome can be 2 kb or more in length and have sequence homology with the target genomic DNA sequence sufficient to allow homologous recombination.

The invention provides cell compositions where the cells contain a site in the genome containing two lox sites. The lox sites can be, for example, a loxP site or a lox511 site. The cells can also contain two non-identical lox sites.

The invention further provides a cell composition comprising a population of non-yeast eukaryotic cells containing a population of 10 or more variant nucleic acids, each of the variant nucleic acids being expressed in a different cell and integrated in the genome of each cell by a site specific recombination sequence. The recognition sequence can be, for example, the 13 amino acid sequence recognized by Cre recombinase.

The cell compositions contain variant nucleic acids or heterologous nucleic acid fragments that are complete and have integrity in that the nucleic acids are the same as those introduced into the cells. The cell compositions exclude those cells containing nucleic acids that are incomplete, for example, cells in which deletions or insertions have occurred in the nucleic acids in vivo, that is, other than those expressly introduced to generate a variant nucleic acid.

The doublelox targeting approach allows the rapid replacement of a chromosomal segment with exogenous transfected DNA in a precisely controlled manner and is an efficient approach for expressing combinatorial protein libraries in mammalian cells. To demonstrate the use of Cre-mediated targeted insertion for the application of directed evolution in mammalian cells, combinatorial protein libraries of the bleomycin resistance protein (BRP) were expressed in mammalian cells, sequenced, and screened as a model system (see Example X). Cre-mediated and Flp-mediated targeted insertion was also demonstrated for libraries of butyrylcholinesterase variants (see Example XI).

BRP is a 14 kDa protein functionally expressed in eukaryotic cells that binds and confers resistance to bleomycin (Gatignol et al., FEBS Lett. 230:171-175 (1988)). Crystallographic data and site-directed mutagenesis studies have identified BRP residues potentially involved in sequestering bleomycin (Dumas et al., EMBO J. 13:2483-2492 (1994)). Thus, BRP possesses ideal characteristics as a model protein for demonstrating the application of directed evolution in mammalian cells. Specifically, the functional activity of BRP is easily measured in eukaryotic cells, and structural information, though not required, is available to permit mutagenesis to be focused on discreet regions of the protein.

Butyrylcholinesterase variants were also generated and expressed in mammalian cells. Cholinesterases are ubiquitous, polymorphic carboxylase Type B enzymes capable of hydrolyzing the neurotransmitter acetylcholine and numerous ester-containing compounds. Two major cholinesterases are acetylcholinesterase and butyrylcholinesterase. Butyrylcholinesterase catalyzes the hydrolysis of a number of choline esters as shown:

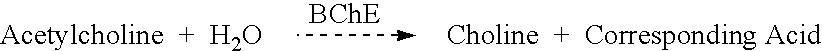

Butyrylcholinesterase preferentially uses butyrylcholine and benzoylcholine as substrates. Butyrylcholinesterase is found in mammalian blood plasma, liver, pancreas, intestinal mucosa and the white matter of the central nervous system. The human gene encoding butyrylcholinesterase is located on chromosome 3, and over thirty naturally occuring genetic variations of butyrylcholinesterase are known. The butyrylcholinesterase polypeptide is 574 amino acids in length and encoded by 1,722 base pairs of coding sequence. Naturally occurring human butyrylcholinesterase variations, species variations, as well as recombinantly prepared mutations have previously been described by Xie et al., Molecular Pharmacology 55:83-91 (1999).

As disclosed herein, the invention provides methods useful for establishing a general and broadly applicable system for the expression of combinatorial protein libraries in mammalian cells. The methods of the invention are applicable in directed evolution technologies in a non-yeast eukaryotic expression system, including a mammalian expression system, as demonstrated by modifying the function of BRP, a protein selected as a model for testing methods of identifying variants having optimized activity (see Examples VII, IX and X), and butyrylcolinesterase (see Example XI).

The invention variant nucleic acids or heterologous nucleic acids can be expressed in a variety of eukaryotic cells. For example, the nucleic acids can be expressed in mammalian cells, insect cells, plant cells, and non-yeast fungal cells. One skilled in the art can readily distinguish a non-yeast fungus such as a mold from a yeast based on well known distinguishing structural and physiological characteristics.

The invention also provides a method of identifying a polypeptide exhibiting optimized activity. The method includes the steps of screening an invention cell composition for an activity associated with a parent polypeptide of a diverse population of variant polypeptides encoded by the variant nucleic acids; and identifying a variant polypeptide exhibiting an optimized activity relative to the parent polypeptide. The methods can therefore be used to identify a polypeptide having an optimized activity. The methods of the invention can similarly be applied to identify a nucleic acid having an optimized activity by screening for an activity associated with a parent nucleic acid. For example, BRP variants having optimized activity for both increased binding and decreased binding activity were identified (see Example X).

The invention additionally provides a method of identifying a binding ligand. The method includes the steps of contacting an invention cell composition with one or more ligands; and identifying a ligand that binds to one of the variant nucleic acids. The invention further provides a method of identifying a binding ligand. The method includes the steps of contacting an invention cell composition with one or more ligands, the cells containing a diverse population of variant polypeptides encoded by the variant nucleic acids; and identifying a ligand that binds to a polypeptide encoded by the variant nucleic acids.

The invention provides a method for determining binding of a receptor to one or more ligands by contacting a receptor variant population with one or more ligands and detecting binding of one or more ligands to the collective receptor variant population. The receptor variant population can be a collective population. The methods of the invention employ a collective population of variant but similar molecules to screen one or more binding partners for a detectable interaction. For example, a collective receptor variant population is screened with one or more ligands to determine binding activity. Using a receptor variant population is advantageous in that the receptor variant population provides an expanded receptor target range compared to a single receptor of similar function for the identification of binding ligands. This expanded target range increases the probability that at least one ligand in a population will have detectable binding affinity for a receptor variant.

Increased probability of detecting binding ligands to a population of variant receptors has practical applications in that a large number of different ligands can be screened with a single variant population to rapidly identify a subset of the ligand population that is most likely to have desired binding properties toward the preferred or parent receptor. Essentially, the use of a population of variant receptors to identify binding partners eliminates in an initial screen ligands that are unlikely to bind the parent receptor. The subpopulation of ligands that exhibit binding to the variant receptor population can be subsequently tested for binding activity and affinity toward the parent receptor. Moreover, if the initial subpopulation of ligands remains relatively large, further screens using subpopulations of variant receptors that reduce the receptor target binding range to variants more closely related to the parent receptor can be performed to narrow the likely binding ligands that exhibit preferential binding characteristics.

In addition to rapidly identifying binding ligands that have a high probability of binding to a desired receptor, the use of an expanded binding target range similarly allows for the rapid identification of a receptor that binds to a particular ligand. In this case, a population of receptors can be screened with a ligand variant population in a similar fashion to that described above in which the receptors which are unlikely to bind to the parent ligand are eliminated. Similarly, the ligand binding range can be reduced by subsequently using ligand variants that are more closely related to the parent ligand so as to preferentially identify receptors that exhibit desired binding characteristics.

Screening variant populations of receptors or ligands to rapidly identify likely binding partners has the added advantage that such a screen will also identify a greater range of binding candidates, including binding partners that exhibit low or undetectable binding toward the parent molecule. For example, the increased probability of detecting a ligand interaction with a receptor variant population can be exemplified in the context of complementary interactions between receptors and ligands. For example, the affinity of a ligand for a receptor can be determined by the chemical functional groups at the site of contact between the receptor and ligand and the relative position of the chemical groups in three-dimensional space. Receptor variants and ligand variants can, for example, differ in chemical functional groups in their contact sites or differ in other chemical functional groups that contribute to the conformation and three-dimensional orientation of the chemical functional groups in the contact site. A receptor variant population contains receptor variants that can differ in the ligand contact site or sites and therefore can have different affinities for different ligands. A ligand can have an affinity for the parent receptor below the level of detectable binding. In contrast, the same ligand can exhibit detectable and even strong binding affinity for a receptor variant. Screening the ligand against the parent receptor would not allow the identification of the ligand as a binding partner. Using a receptor variant population therefore increases the likelihood of identifying ligands that bind to the parent receptor regardless of affinity. Having the capability of identifying ligands independent of its binding strength allows the selection of a ligand exhibiting a relative affinity suitable for an intended purpose.

In addition, screening with a receptor variant population provides additional information about the relative affinity of a given binding ligand for a target receptor. For example, a ligand that binds to a larger number of receptor variants has an increased likelihood of binding to the target or parent receptor than one that binds to fewer receptor variants such as only one receptor variant. Thus, more information is obtained when ligands are screened with a receptor variant population than when ligands are screened with the parent receptor alone.

Additionally, the binding ligands identified using methods of the invention can be used to generate a library of ligand variants. The identified ligand is used as a parent ligand to generate a library containing a ligand variant population. The library of ligand variants can be based on structural similarities to the parent ligand, for example, such libraries of ligand variants can be generated using combinatorial chemistry methods (Combinatorial Peptide and Nonpeptide Libraries: A Handbook, Jung, ed., VCH, New York (1996); Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997); Terrett, Combinatorial Chemistry, Oxford University Press, New York (1998); Czarnik and DeWitt, eds., A Practical Guide to Combinatorial Chemistry, American Chemical Society, Washington DC (1997)).

The characteristics of the receptor variants can be varied depending on the needs of a particular ligand screen. For example, if the receptor variants are closely related, then a ligand that binds to the most number of receptor variants has the greatest likelihood of binding to the parent receptor. The characteristics of the receptor variants can also be varied so that the receptor variants in a population are less closely related. Thus, depending on the needs of the investigator, the receptor variants can be made to be more or less closely related.

The relatedness of the receptor variant to the parent receptor can be determined by the chemical similarities or differences of the particular chemical functional groups that define the receptor variant relative to the analogous chemical functional group in the parent receptor. For example, if the parent receptor or ligand is a polypeptide, the relatedness of the variants to the parent is determined by the relatedness of the amino acids that differ between the variants and the parent molecule. A chemically more conservative difference between the variant and the parent results in variants more closely related to the parent molecule. Conservative substitutions of amino acids include, for example, (1) non-polar amino acids (Gly, Ala, Val, Leu and Ile); (2) polar neutral amino acids (Cys, Met, Ser, Thr, Asn and Gln); (3) polar acidic amino acids (Asp and Glu); (4) polar basic amino acids (Lys, Arg and His); and (5) aromatic amino acids (Phe, Tyr, Trp and His). Additionally, conservative substitutions of amino acids include, for example, substitutions based on the frequencies of amino acid changes between corresponding proteins of homologous organisms (Principles of Protein Structure, Schulz and Schirmer, eds., Springer Verlag, New York (1979)).

A ligand generally interacts with a receptor through multiple molecular interactions resulting from multiple contact points or through multiple interactions of a chemical functional group that can be described, for example, as three points. These three points can be, for example, three distinct chemical groups that serve as contact points for the binding partner. Likewise, three different amino acids or three different clusters of amino acids in a polypeptide ligand or receptor can serve as contact points for the binding partner. In this case, binding between the ligand and receptor will occur only when all three points can bind.

Using the above multiple-point binding description for ligand-receptor interactions, a receptor variant population can be generated in which one of the points is fixed so that it is identical to the parent receptor and the other points are varied to generate a receptor variant population. For example, using three reference points, one point is fixed to be identical to the parent receptor and the other two points are varied to generate a receptor variant population. By generating a receptor variant population, the probability of detecting binding of a ligand to one of the receptor variants is increased. Identification of a binding ligand can then be performed as an iterative process. A ligand identified by fixing one point and varying the other contact points on the receptor can be used to generate a library of ligand variants. In the next iteration of the process, the original receptor contact point can be fixed and an additional point can be fixed to be identical to the parent receptor. In the example above describing three reference points, two points are fixed to be identical to the parent receptor and one point is varied to generate a second receptor variant population. The library of ligand variants is screened with the second receptor variant population to identify binding ligands from the ligand variant library. The binding activity of the identified binding ligands can be compared to identify a ligand variant having optimal binding activity to the parent receptor. The process of fixing additional receptor contact points, identifying one or more ligand variants with optimal binding and generating a library of ligand variants is repeated until a ligand is identified that binds to the parent receptor with optimal activity. Thus, a population of ligands or a population of ligand variants can be screened with different receptor variant populations derived from the same parent receptor to identify binding ligands.

A parent receptor can be any molecule that binds to a ligand. The receptors can be, for example, cell surface receptors that transmit intracellular signals upon binding of a ligand. For example, the G protein coupled receptors span the membrane seven times and couple signaling to intracellular heterotrimeric G proteins. G protein coupled receptors participate in a wide range of physiological functions, including hormonal signaling, vision, taste and olfaction. Moreover, these receptors encompass a large family of receptors, including receptors for acetylcholine, adenosine and adenine nucleotides, β-adrenergic ligands such as epinephrine, angiotensin, bombesin, bradykinin, cannabinoids, chemokines, dopamine, endothelin, histamine, melanocortins, melanotonin, neuropeptide Y, neurotensin, opioid peptides, platelet activating factor, prostanoids, serotonin, somatostatin, tachykinin, thrombin and vasopressin, among others.

Other cell surface receptors have intrinsic tyrosine kinase activity and include growth factor or hormone receptors for ligands such as platelet-derived growth factor, epidermal growth factor, insulin, insulin-like growth factor, hepatocyte growth factor, and other growth factors and hormones. In addition, cell surface receptors that couple to intracellular tyrosine kinases include cytokine receptors such as those for the interleukins and interferons.

Integrins are cell surface receptors involved in a variety of physiological processes such as cell attachment, cell migration and cell proliferation. Integrins mediate both cell-cell and cell-extracellular matrix adhesion events. Structurally, integrins consist of heterodimeric polypeptides where a single a chain polypeptide noncovalently associates with a single β chain. In general, different binding specificities are derived from unique combinations of distinct α and β chain polypeptides. For example, vitronectin binding integrins contain the αᵥ integrin subunit and include αᵥβ₃, αᵥβ₂ and αᵥβ₅, all of which exhibit different ligand binding specificities.

Receptors also can function in the immune system. An antibody or immunoglobulin is an immune system receptor which binds to a ligand. The polypeptide receptor can be the entire antibody or it can be any functional fragment thereof which binds to the ligand. Functional fragments such as Fab, F(ab)₂, Fv, single chain Fv (scFv) and the like are included within the definition of the term antibody. The use of these terms in describing functional fragments of an antibody are intended to correspond to the definitions well known to those skilled in the art. Such terms are described in, for example, Harlow and Lane, Antibodies: A Laboratory Manual, Cold Spring Harbor Laboratory, New York (1989), which is incorporated herein by reference.

As with the above terms used for describing antibodies and functional fragments thereof, the use of terms which reference other antibody domains, functional fragments, regions, nucleotide and amino acid sequences and polypeptides or peptides, is similarly intended to fall within the scope of the meaning of each term as it is known and used within the art. Such terms include, for example, “heavy chain polypeptide” or “heavy chain”, “light chain polypeptide” or “light chain”, “heavy chain variable region” (V_{H}) and “light chain variable region” (V_{L}) as well as the term “complementarity determining region” (CDR).

In addition to antibodies, the receptors can be T cell receptors (TCR). T cell receptors contain two subunits, α and β, which are similar to antibody variable region sequences in both structure and function. In this regard, both subunits contain variable region which encode CDR regions similar to those found in antibodies (Immunology, Third Ed., Kuby, J. (ed.), New York, W.H. Freeman & Co. (1997)). The CDR containing variable regions of TCRs bind to antigens presented on the cell surface of antigen-presenting cells and are capable of exhibiting binding specificities to essentially any particular antigen.

Other exemplary receptors of the immune system which exhibit known or inherent binding functions include major histocompatiblility complex (MHC), CD4 and CD8. MHC functions in mediating interactions between antigen-presenting cells and effector T cells. CD4 and CD8 receptors function in binding interactions between effector T cells and antigen-presenting cells. CD4 and CD8 also exhibit similar CDR region structure as do antibodies and TCRs sequences.

The generation of receptor variant populations can be by any means desired by the user. Those skilled in the art will know what methods can be used to generate receptor variants. For example, receptor variants of a given polypeptide receptor can be generated by mutagenesis of one or more amino acids in functional domains so long as the receptor variant retains a structural or functional similarity to the parent receptor. In such a case, mutagenesis of the receptor can be carried out using methods well known to those skilled in the art (Molecular Cloning: A Laboratory Manual, Sambrook et al., eds., Cold Spring Harbor Press, Plainview, N.Y. (1989)). For example, in the case of G protein coupled receptors, the extracellular domain can be identified based on sequence homology and topology of the seven membrane spanning domains of this class of receptors. Mutagenesis of the regions corresponding to the extracellular domain can provide a receptor variant population useful for screening ligands that bind to and elicit a signaling response from the parent G protein coupled receptor.

One method well known in the art for rapidly and efficiently producing a large number of alterations in a known amino acid sequence or for generating a diverse population of random sequences is known as codon-based synthesis or mutagenesis. This method is the subject matter of U.S. Pat. Nos. 5,264,563 and 5,523,388 and is also described in Glaser et al. J. Immunology 149:3903-3913 (1992). Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. For the synthesis of equal frequencies of all twenty amino acids, up to two codons can be synthesized in each reaction vessel.

Variations to these synthesis methods also exist and include for example, the synthesis of predetermined codons at desired positions and the biased synthesis of a predetermined sequence at one or more codon positions. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of XXG/T nucleotides where X is a mixture of all four nucleotides. Following synthesis of the predetermined and random codons, the reaction products in each of the two reaction vessels are mixed and then redivided into an additional two vessels for synthesis at the next codon position.

A modification to the above-described codon-based synthesis for producing a diverse number of variant sequences can similarly be employed for the production of the variant populations described herein. This modification is based on the two vessel method described above which biases synthesis toward the parent sequence and allows the user to separate the variants into populations containing a specified number of codon positions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds by then dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three and four positions as well as others can be conveniently separated out and used based on the need of the individual. Moreover, this synthesis scheme also allows enrichment of the populations for the randomized sequences over the parent sequence since the vessel containing only the parent sequence synthesis is similarly separated out from the random codon synthesis.

The efficient synthesis and expression of libraries of antibody variants synthesized using oligonucleotide-directed mutagenesis can be synthesized as previously described (Wu et al., Proc. Natl. Acad. Sci. USA, 95:6037-6042 (1998); Wu et al., J. Mol. Biol., 294:151-162 (1999); Kunkel, Proc. Natl. Acad. Sci. USA, 82:488-492 (1985)). Oligonucleotide-directed mutagenesis is a well-established and efficient procedure for systematically introducing mutations, independent of their phenotype and is, therefore, ideally suited for directed evolution approaches to protein engineering. The methodology is flexible, permitting precise mutations to be introduced without the use of restriction enzymes, and is relatively inexpensive if oligonucleotides are synthesized using codon-based mutagenesis. Briefly, to perform oligonucleotide-directed mutagenesis, a population of oligonucleotides encoding the desired mutation(s) is hybridized to single-stranded uracil-containing template of the wild type sequence. To generate a single-stranded template containing uracil, the dut⁻ung⁻ E. Coli strain CJ236 (Bio-Rad; Richmond, Calif.) is infected with a plasmid containing a filamentous phage origin of replication (phagemid vector). Super-infection of bacterial cells containing the phagemid results in the production and secretion of single-stranded uracil-containing DNA. Following annealing of the mutagenic oligonucelotide(s) to the uracil template, T4 DNA polymerase, dNTP, and T4 DNA ligase are added to generate double-stranded circular DNA, and the mutant DNA is efficiently recovered following transformation of a dut⁺ ung⁺ bacterial strain.

Populations of variants can also be generated using gene shuffling. Gene shuffling or DNA shuffling is a method for directed evolution that generates diversity by recombination (see, for example, Stemmer, Proc. Natl. Acad. Sci. USA 91:10747-10751 (1994); Stemmer, Nature 370:389-391 (1994); Crameri et al., Nature 391:288-291 (1998); Stemmer et al., U.S. Pat. No. 5,830,721, issued Nov. 3, 1998). Gene shuffling or DNA shuffling is a method using in vitro homologous recombination of pools of selected mutant genes. For example, a pool of point mutants of a particular gene can be used. The genes are randomly fragmented, for example, using DNase, and reassembled by PCR. If desired, DNA shuffling can be carried out using homologous genes from different organisms to generate diversity (Crameri et al., supra, 1998). The fragmentation and reassembly can be carried out in multiple rounds, if desired. The resulting reassembled genes are a library of variants that can be used in the invention compositions and methods.

Methods for preparing libraries containing diverse populations of various types of molecules such as peptides, peptoids and peptidomimetics are well known in the art (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide chemistry are well known in the art.

Populations of receptor variants can be alternatively derived from a family of related receptors. Again using G protein coupled receptors as an example, a receptor variant population can be a collection of G protein coupled receptor family members. Because these proteins are structurally similar and carry out similar functions, they constitute a family of structurally related receptor variants that function in ligand binding. Such a receptor family can be isolated using available sequence information on the receptors and generating primers that can amplify the receptor family or generating probes that can be used to isolate genes of the family members.

In addition, a population of receptor variants can be generated from a family of related receptors even when all members of the family have not been identified. In this case, a receptor of interest is identified and related family members are isolated by, for example, generating probes that allow isolation of the related family members or by generating primers that hybridize with conserved structural domains of the parent receptor and amplifying related family members.

To obtain cells capable of targeting a nucleic acid to an identical site in the genome, a recombination sequence can be incorporated into the genome of a cell. For example, a recombination sequence can be targeted to a site in the genome by transfecting a vector containing a recombination sequence and isolating clones, as described previously ((Bethke and Sauer, Nuc. Acids Res., 25:2828-2834 (1997)). The clones can be screened for low copy number or single copy number, and an individual clone can be used to target nucleic acids flanked by homologous site-specific recombinase recognition sequences. In addition, a sequence useful for homologous recombination using endogenous recombination machinery can similarly be obtained by transfection and isolation of clones, as described above.

In order to use recombinase-mediated targeted insertion as a general approach for applying directed evolution technologies in mammalian cells, it is desirable to achieve efficient transfection so that libraries containing thousands of distinct protein variants can be easily expressed. Efficient transfection and targeted integration can be achieved by varying the method of introducing the DNA into the cells, the amount of the targeting vector encoding variant nucleic acids or heterologous nucleic acid fragments, and/or the total mass of DNA used per transfection. If the target vector encoding variant nucleic acids or heterologous nucleic acid fragments are co-transfected with a recombinase expression vector, the ratio of targeting vector and recombinase vector can be varied.

Previously, a variety of transfection methods have been used to introduce the targeting vector into different host lines. For example, 13-1 cells have been transfected using calcium phosphate (Bethke and Sauer, Nuc. Acids Res., 25:2828-2834 (1997), while the lox target cell line 14-1-2 has been transfected using lipofection (Fukushige and Sauer, Proc. Natl. Acad. Sci. USA 89:7905-7909 (1992); Baubonis and Sauer, Nuc. Acids Res., 21:2025-2029 (1993)). The mechanisms mediating DNA transfection by calcium phosphate (Chen and Okayama, Mol. Cell. Biol., 7:2745-2752 (1987)) and liposomes are not precisely understood but are likely to be distinct. Therefore, the transfection parameters can be varied by cell type and optimized empirically (see Example VIII). Furthermore, it is understood that introduction of the targeting vector can be achieved by both stable or transient cell transfection.

The results disclosed herein demonstrate the feasibility of expressing and screening a library of protein variants in non-yeast eukaryotic cells such as mammalian cells (see Examples X and XI). The approach is general and can be applied to any protein expressed functionally in eukaryotic cells. An important aspect for applying this approach broadly is the 0.5% efficiency of the targeted integration routinely obtained (see Example VIII). Targeted integration efficiencies of 0.5% permit the use of non-yeast eukaryotic expression libraries such as mammalian expression libraries containing >10,000 unique members simply by transfecting as few as 2×10⁶ host cells. Previously, directed evolution of proteins expressed in bacterial cells has been used to engineer desired characteristic(s) of the protein of interest by synthesizing libraries containing −3,000 unique variants. The methods disclosed herein using cultured non-yeast eukaryotic cells such as mammalian cells provide a more relevant environment for engineering proteins for therapeutic use than use of bacterial cells because of the compartmentalization and post-translational modifications unique to mammalian cells. Therefore, the non-yeast eukaryotic cell expression system including the eukaryotic cell system disclosed herein can be used for engineering proteins that can be expressed in bacterial cells.

Using the methods disclosed herein, a population of non-yeast eukaryotic cells containing a diverse population of variant nucleic acids or heterologous nucleic acid fragments can be generated routinely and reproducibly without further characterization of the accuracy of intergration. Therefore, after introducing variant nucleic acids or heterologous nucleic acid fragments into cells to generate a population of cells, the population can be used directly for screening without further characterization of the cells. However, further characterization of the cells containing variant nucleic acids or heterologous nucleic acid fragments can be performed, if desired.

It is understood that the methods disclosed herein directed to receptor variants can similarly be applied to screen for activities other than binding activity. The methods can be used to screen for any activity that can be measured, for example, a biological activity or enzymatic activity.

Once a receptor has been identified and a variant receptor population has been generated, the receptor variants are produced in a manner convenient for detecting ligand binding to-a collective receptor variant population. One such system involves expressing receptor variants in cells such that binding of ligands to the receptor variants can be detected in culture. One detection method is based on utilizing the cellular signaling properties of the receptor to detect binding of a ligand. Utilizing the signaling properties of the receptor variants is convenient because it allows detection of ligand binding without the need to isolate and purify the receptor variant population or to prepare cell extracts for in vitro assays.

One system for detecting cellular signaling events is the melanophore system (Lerner, Trends Neurosci. 17:142-146 (1994)). Melanophores are skin cells that provide pigmentation to an organism. The equivalent cells in humans are melanocytes, which are responsible for skin and hair color. In numerous animals, including fish, lizards and amphibians, melanophores are used, for example, for camouflage. The color of the melanophore is dependent on the intracellular position of melanin-containing organelles, called melanosomes. Melanosomes move along a microtubule network and are clustered to give a light color or dispersed to give a dark color. The distribution of melanosomes is regulated by G protein coupled receptors and cellular signaling events, where increased concentrations of second messengers such as cyclic AMP and diacylglycerol results in melanosome dispersion and darkening of the melanophores. Conversely, decreased concentrations of cyclic AMP and diacylglycerol results in melanosome aggregation and lightening of the melanophores.

The level of second messengers is regulated by hormones. Melatonin stimulates receptors that lower intracellular second messenger levels and thus causes the cells to lighten. In contrast, melanocyte stimulating hormone (MSH) increases intracellular second messenger levels and causes the melanophores to darken. Other regulators of melanosome distribution include catecholamines, endothelins and light. Thus, cells darken in response to photostimulation.

The melanophore system is advantageous for testing receptor-ligand interactions including G protein coupled receptors due to the regulation of melanosome distribution by receptor stimulated intracellular signaling. For example, a G protein coupled receptor can be selected as the parent receptor and a receptor variant population can be generated. The receptor variant population is transfected into melanophore cells, for example, frog melanophore cells, and the G protein coupled receptor variants are expressed. Ligands that stimulate or inhibit G protein coupled receptor signaling can be determined since the system can be used to detect both aggregation of melanosomes and lightening of cells and dispersion of melanosomes and darkening of cells.

In addition to G protein coupled receptors, the melanophore system is also useful for testing other tinges of receptors so long as the receptors couple into a signaling mechanism that regulates melanosome distribution. For example, many receptor tyrosine kinases couple to changes in diacylglycerol. Since diacylglycerol is a second messenger that regulates melanosome distribution, ligands that function as agonists or antagonists of these receptors or that stimulate or inhibit their tyrosine kinase activity can be analyzed using the melanophore system.

In addition to the melanophore system, other systems can be used to detect signaling events of receptors. Receptors often initiate intracellular signaling events that induce the expression of early response genes. For example, many receptor tyrosine kinases induce the early response gene fos. A reporter system can be generated, for example, by fusing the fos promoter to a detectable protein such as luciferase. Ligands that stimulate or inhibit cellular signaling from these receptors can be detected using the endogenous cellular signaling machinery without the need to perform time consuming in vitro assays.

A collective receptor variant population is contacted with one or more ligands by incubating the ligands under conditions that allow binding. For example, the ligands can be contacted and incubated with the collective receptor variant population under conditions similar to physiological conditions, such as incubation in isotonic solution at 37° C. Unbound ligands are removed from the collective receptor variant population and binding of ligands to receptor variants is detected. For example, the darkening or lightening of melanophore cells can be used to detect binding of a ligand to a receptor variant.

The invention provides methods for contacting a collective receptor variant population with one or more ligands and detecting ligand binding to the collective receptor variant population. An additional advantage of screening a collective receptor variant population is that, unlike traditional screening methods, which require that the population be segregated such that individual members can be identified, the present invention screens the receptor variant population as a non-segregated pool. The collective receptor population provides an advantage in that a collective receptor population significantly reduces the surface area or volume required to contact the collective receptor population with ligands, thereby increasing the capacity to screen many more ligands for binding interactions.

The invention provides methods for dividing the collective receptor variant population into two or more subpopulations, contacting one or more of the receptor variant subpopulations with one or more ligands and detecting one or more receptor variant subpopulations having binding activity to one or more ligands. One of the receptor variant subpopulations, all of the receptor variant subpopulations or an intermediate number of receptor variant subpopulations can be screened.

For example, a particular collective receptor known to give a large number of binding interactions. In this example, it is sufficient to contact a receptor variant subpopulation rather than the entire receptor variant population to identify a ligand binding to a receptor variant. One skilled in the art knows how many receptor variant subpopulations are sufficient to provide a likely probability of detecting ligand binding activity given the teachings described herein. After detecting binding of one or more ligands to a collective receptor variant population, the collective receptor variant population is divided into two or more subpopulations and contacted with the ligand or ligands. The receptor variant subpopulations can be collective when two or more receptor variants are in the subpopulation. The receptor variant subpopulations need not contain equal numbers of receptor variants. At least one of the receptor variant subpopulations will bind to the ligand or ligands, although more than one receptor variant subpopulation can be detected if more than one receptor variant binds to the ligand or ligands.

The invention also provides methods for repeating the dividing, contacting and detecting one or more times. Once binding has been detected, one or more receptor variants can be determined to have binding activity to one or more ligands. Such a determination allows identification of ligand binding activity to a receptor that can be optimal binding activity. The identification of individual receptor variants with binding to the ligand or ligands is accomplished when the receptor variant subpopulation is repeatedly divided and tested for binding activity until the receptor variant that binds to one or more ligands.

Alternatively, individual receptor variants with binding to one or more ligands can be identified without dividing receptor variant subpopulations into subpopulations containing only a single receptor variant. Individual receptor variants in a collective receptor variant population can be identified using a system for tagging receptor variants. One approach is to synthesize a tag that is correlated with the generation of receptor variants. For example, a receptor variant population can be generated by mutagenizing a region of the parent receptor. While mutagenizing the receptor to generate receptor variants, a tag specific for that mutant can be generated in parallel. For example, peptides that are expressed on the surface of cells and that are recognized by specific antibodies can be used as tags to identify a co-expressed receptor variant.

Introduction of mutations that generate receptor variants can be performed, for example, using the codon-based synthesis methods described herein. Alternatively, mutations can be introduced by excising the region of the receptor cDNA to be mutagenized from a parent vector. In parallel, the region corresponding to the peptide tag can be excised as well. Mutation of a specific amino acid or amino acids in the parent receptor can be correlated with a specific mutation of one or more amino acids in the peptide to generate a unique peptide recognized by, for example, a specific antibody. The DNA fragment containing the mutated residues can be inserted into the parent vector to introduce these mutations into the receptor and the peptide tag. Appropriate restriction enzyme sites can be used to allow cloning, or loxP sites can be used to allow site-specific recombination into the parent vector. Thus, a specific receptor variant is correlated with a specific peptide tag.

In the specific example of the melanophore expression system described above, a positive cell expressing a receptor variant that binds to a ligand can be isolated from other cells in the population by cell sorting using dark and light properties of the melanophore cells. The isolated positive cell can then be analyzed with respect to the peptide tag expressed on its cell surface. Identification of the peptide tag allows identification of the receptor variant that binds the ligand.

A sufficiently large number of tags can be generated with a limited number of different peptides and antibodies specific for those peptides. This can be accomplished by restricting specific peptides to specific positions. For example, a combination of 32 different peptides can be used to generate 4096 (8⁴) different tags by restricting 8 specific peptides to 4 specific positions.

The tag system can be used to isolate and identify individual receptor variants in a collective receptor variant population that binds to a ligand or ligands. For example, a cell surface expressed tag consisting of peptides can be identified using antibodies specific for the peptides in fluorescence activated cell sorting (FACS) analysis. Individual receptor variants can be isolated using the unique tag associated with each receptor variant. In addition, because the tag is coordinated with a specific receptor variant, the individual receptor variant can be identified. In the case where 32 peptide and antibody combinations are used to generate 4096 different tags, exposing the cells to each of the 32 antibodies in FACS analysis allows the isolation and identification of individual receptor variants. The number of individual receptor variants that binds to the ligand or ligands can be used to identify an optimal binding ligand and can give an indication of the efficaciousness of the ligand as a lead compound for drug development.

The methods and compositions disclosed herein directed to variant nucleic acids can also be applied to the expression of heterologous nucleic acids in a population of cells. The invention also provides a cell composition comprising a population of non-yeast eukaryotic cells containing a diverse population of 10 or more heterologous nucleic acid fragments, the heterologous nucleic acid fragments comprising distinct species of nucleic acid fragments and each of the heterologous nucleic acid fragments being expressed in a different cell and located within each cell at an identical site in the genome. The invention additionally provides methods of using a population of cells containing heterologous nucleic acid fragments to identify binding ligands, similar to the methods disclosed herein directed to cells containing variant nucleic acids.

The invention also provides a method of identifying a polypeptide receptor for a ligand. The methods include the steps of contacting a population of non-yeast eukaryotic cells containing a diverse population of 10 or more heterologous nucleic acid fragments encoding polypeptides with a ligand, the heterologous nucleic acid fragments comprising distinct species of nucleic acid fragments, each of the heterologous nucleic acid fragments being expressed in a different cell and located within each cell at an identical site in the genome; and identifying a polypeptide encoded by the heterologous nucleic acid fragments that binds to the ligand.

The invention further provides a method of identifying a functional polypeptide fragment. The methods include the steps of introducing a diverse population of 10 or more heterologous nucleic acid fragments into a non-yeast eukaryotic cell to generate a population of cells, the heterologous nucleic acid fragments comprising distinct species of nucleic acid fragments, each of the nucleic acid fragments being expressed in a different cell and located within each cell at an identical site in the genome; screening the population of cells for a functional activity; and identifying a polypeptide encoded by said nucleic acid fragments having said functional activity.

Exemplary functional activities include binding, catalysis, biological activity, or any type of functional activity. It is understood that any measurable activity useful for identifying a polypeptide encoded by a nucleic acid fragment can be used in methods of the invention. Methods for screening for a functional activity of a polypeptide encoded by a heterologous nucleic acid fragment are well known to those skilled in the art, including the well known methods of expression screening (see Ausubel et al., Current Protocols in Molecular Biology (Supplement 47), John Wiley & Sons, New York (1999)). For example, a population of cells containing a diverse population of heterologous nucleic acid fragments can be screened for binding activity to a ligand such as a small molecule, polypeptide or antibody. Such a binding assay can be performed on whole cells or cell lysates, if desired. When assaying intact cells, the polypeptide encoded by the heterologous nucleic acid fragment can be expressed on the cell surface and accessible to the ligand or the ligand can have a chemical composition that allows it to be specifically taken up by the cell or to penetrate the membrane, thereby being accessible to intracellularly expressed polypeptides.

In addition, catalytic activity can be measured by screening for an enzymatic activity using whole cells or cell lysates. Any catalytic activity for which an enzymatic assay can be performed can be used to screen a population of cells containing heterologous nucleic acid fragments to identify a polypeptide encoded by a nucleic acid fragment having the functional activity. Such catalytic activities can be classified as oxireductase, transferase, hydrolase, lyase, isomerase and ligase. Specific examples of catalytic activities for which an assay can be performed include, but are not limited to, kinase, GTPase, and phosphatase.

Cells expressing heterologous nucleic acid fragments can also be screened for a biological activity. For example, cells can be screened for the effect of polypeptides encoded by the heterologous nucleic acid fragments on a signaling pathway such as the G-protein coupled receptor-based assays disclosed herein or any of the well known signaling pathways such as the MAP kinase pathway, steroid hormone receptor pathway, or any signaling pathway. It is understood that, similar to the screening of catalytic activity as disclosed herein, screening assays can be performed for a wide range of signaling pathways known to those skilled in the art.

A biological activity can also be monitored using a reporter gene assay. Such reporter gene assays and systems are well known to those skilled in the art (Ausubel et al., supra, 1999). A reporter gene assay can be used to monitor alterations in a signaling pathway associated with the reporter gene assay, for example, signaling pathways that alter gene expression of the reporter gene. A polypeptide encoded by a nucleic acid fragment that alters a signaling pathway associated with the reporter gene can be detected by changes in reporter gene expression.

The methods of the invention directed to expression of heterologous or variant nucleic acids in non-yeast eukaryotic cells are particularly useful for screening polypeptides, which often do not fold properly in the environment of a bacterial cell or which undergo postranslational modification in eukaryotic cells. Thus, the methods of the invention are particularly advanatageous for screening eukaryotic polypeptides that are folded and processed in a eukaryotic environment, The methods are also useful because a polypeptide can be tested for its effect on a signaling pathway in a eukaryotic environment since such signaling pathways are generally absent in a bacterial cell.

Furthermore, the methods can be performed in a cell line having a particular gene deleted. Such a cell line can be used to screen for a polypeptide encoded by a nucleic acid fragment that substitutes for the deleted activity or compensates for the deleted activity. For example, a polypeptide can substitute for a deleted activity by providing a similar activity. Such a method can be used, for example, to screen for other polypeptides having a similar activity or to identify species equivalents of a deleted gene. A polypeptide can also compensate for a deleted activity, for example, by altering another polypeptide in a signaling pathway associated with the deleted gene. Therefore, the methods of the invention can be used to identify a polypeptide encoded by a heterologous nucleic acid fragment that functions in or alters a signaling pathway.

Similar assays to those described above for identifying a polypeptide encoded by a heterologous nucleic acid fragment having-a functional activity can also be applied to screening or determining an activity of a polypeptide encoded by a variant nucleic acid. For example, a cell line can be generated having a particular gene deleted, and variants of that gene can be introduced into the cell and screened for an activity. Such a cell line can be useful for reducing the background signal of a particular activity associated with a nucleic acid or encoded polypeptide for which a variant population has been generated.

Furthermore, the methods can be performed to screen for functional activity that occurs in response to a particular signaling pathway. For example, libraries can be screened on live cells where the expected response to such signaling is cell proliferation or cell death. Any signaling pathway for which an effect can be measured can be used as a screen for functional activity.

The invention also provides a method for determining binding of a ligand to one or more receptors by contacting a collective ligand variant population with one or more receptors and detecting binding of one or more receptors to the collective ligand variant population. The invention further provides a method for dividing the collective ligand variant population into two or more subpopulations, contacting one or more of the two or more subpopulations with one or more receptors and detecting one or more ligand variant subpopulations having binding activity to one or more receptors.

Methods and procedures described above for determining binding of a receptor to one or more ligands can similarly be applied to determine the binding of a ligand to one or more receptors. As described herein, methods are provided for repeating the dividing of ligand variant population or subpopulations, contacting with one or more receptors and detecting-binding activity. Furthermore, detection of ligand binding activity allows identification of a ligand variant having binding activity to one or more receptors. Optimal binding activity can be determined relative to a predetermined standard. For example, the ligand with optimal binding can be the ligand that binds to one or more receptors at the highest affinity. Alternatively, optimal binding can be binding to the largest number of receptor variants or binding to greater than some threshold number of receptor variants.

The invention additionally provides a method for determining binding of a ligand to a receptor or variant thereof by contacting a collective ligand population with the receptor or variant thereof and detecting binding of the receptor or variant thereof to the collective ligand population.

The collective ligand population, which can be structurally related ligand variants or can be unrelated structurally, is contacted with a parent receptor or one or more receptor variants. For example, the parent receptor and receptor variants can be expressed in an appropriate cell line such as the melanophore cell line. The collective ligand population is contacted with the parent or one or more receptor variants and binding of one or more ligands in the collective ligand population is detected, for example, by detecting a change in melanophore cell color.

The invention additionally provides methods for dividing the collective ligand population into two or more subpopulations, contacting one or more of the two or more subpopulations with the receptor or variant thereof and detecting one or more ligand subpopulations with binding activity to the receptor or variant thereof. The ligand subpopulations can contain an unequal number of ligands.

The invention further provides methods for repeating the dividing, contacting and detecting one or more times. The ligand population can be divided until the subpopulation contains a single ligand. Detection of ligand binding activity allows identification of a ligand variant having binding activity to the receptor or variant thereof. An individual ligand having optimal binding activity is determined relative to a predetermined standard. A ligand variant population can be expressed in vitro, for example, by synthetic methods, or the ligand variants can be expressed in a population of cells. The ligand variants can be expressed recombinantly using the methods disclosed herein.

The invention also provides a method for identifying an optimal binding ligand variant for a receptor. The method consists of (a) contacting a collective receptor variant population or subpopulation thereof with a ligand population; (b) detecting binding of one or more ligands in the ligand population to the collective receptor variant population or subpopulation thereof; (c) dividing the ligand population into subpopulations; and (d) repeating optionally each of steps (a) to (c), wherein the ligand subpopulation in step (c) comprises two or more ligands and is used as the ligand population in step (a) and wherein the detecting in step (b) identifies one or more ligands having binding activity to the collective receptor variant population.

The method for identifying an optimal binding ligand variant can include the additional steps of (e) generating a library of variants of the ligand identified in step (d); (f) contacting a parent receptor with each of the ligand variants; and (g) detecting the binding of one or more ligand variants to the parent receptor.

Following identification of one or more ligands having binding activity to the collective receptor variant population, the identified ligand can be used as a parent ligand to generate a library of ligand variants with structural similarities to the parent ligand. The library of ligand variants can be, for example, a population of ligand variants that are screened for binding activity to the parent receptor. Once ligand variants having binding activity have been identified, the binding activity of the ligand variants can be further compared to each other or to a predetermined standard. Such a comparison allows identification of a ligand variant having optimal binding activity to a parent receptor.

As described previously in regard to the multiple binding points of reference for ligand-receptor interactions, particular chemical functional groups can be fixed so that they are identical to the parent ligand. Ligand variants with one chemical group fixed differ from the parent ligand at other chemical groups. Following identification of a ligand with optimal binding, a library of ligand variants can be generated and a ligand variant having optimal binding to the parent receptor is determined. The ligand variant with optimal binding to the parent ligand can be used as a second parent ligand to generate a second library of ligand variants. Such ligand variants can have two chemical groups fixed to be identical to the second parent ligand. An iterative process of identifying individual ligands or ligand variants with optimal binding to the parent receptor and generating a new library based on that identified ligand variant can be repeated to determine a ligand variant with optimal binding to the parent receptor. The ligand variants can be identified based on structural or functional criteria or synthesized by various means known to those skilled in the art. Where the ligand is a polypeptide, for example, variants can be made and screened using surface display methods known to those skilled in the art and using, for example, the codon-based synthesis procedures described herein.

The invention also provides a method for identifying an optimal binding ligand variant to a receptor. The method consists of (a) contacting two or more subpopulations of a collective receptor variant population with individual ligands from a ligand population; (b) detecting binding of one or more individual ligands to one or more of the subpopulations of the collective receptor variant population; (c) dividing at least one of the subpopulations of the collective receptor population which exhibits binding activity to the individual ligands into two or more new subpopulations; and (d) repeating optionally each of steps (a) to (c), the two or more new subpopulations in step (c) comprising two or more receptor variants and the new subpopulations used as the two or more subpopulations of a collective receptor variant population in step (a), wherein the detecting in step (b) identifies one or more individual ligands having binding activity to one or more new subpopulations of subpopulations of the collective receptor variant population.

The method for identifying an optimal binding ligand variant can include the additional steps of (e) contacting a closely related receptor variant subpopulation comprising a parent receptor or a closely related variant thereof with one or more individual ligands identified in step (d); (f) detecting binding of one or more individual ligands to the closely related receptor variant subpopulation; and (g) comparing the binding activity of one or more ligands having binding activity to the closely related receptor variant subpopulation, wherein said comparing identifies a ligand having optimal binding activity to the closely related receptor variant subpopulation.

The method for identifying an optimal binding ligand variant to a receptor can also include the additional steps of (h) generating a library of variants of said ligand identified in step (g); (i) contacting said parent receptor with each of said ligand variants; and (j) detecting binding of one or more ligand variants to said parent receptor.

After identifying one or more ligands having binding activity to the collective receptor variant population, the identified one or more ligands can be further used to screen a closely related receptor variant subpopulation containing at least a parent receptor or a closely related variant thereof. The subpopulation can contain any number of receptor variants so long as they are closely related to the parent receptor. One skilled in the art knows the closeness of the relationship of the receptor variants to the parent receptor sufficient to determine an optimal binding ligand. A ligand that binds to the most number of receptor variants in a closely related receptor variant subpopulation will have the greatest probability of binding to the parent receptor and has the greatest likelihood of being an optimal binding ligand. Such an optimal binding ligand can be used as a lead compound for drug development. In contrast, a receptor variant subpopulation containing less closely related receptor variants provides a decreased probability that a ligand that binds to the most number of receptor variants will also bind to the parent receptor.

A ligand having optimal binding activity to the closely related receptor variant subpopulation can be further used as a parent ligand to generate a library of ligand variants with structural similarities to the parent ligand. One skilled in the art knows what optimal binding activity is desired. For example, a ligand having optimal binding activity can be one that binds to the most number of receptor variants in the closely related receptor variant subpopulation. Optimal binding activity also can be defined as ligands that bind to a minimum threshold of numbers of receptor variants. The library of ligand variants can be, for example, a population of ligand variants that are screened for binding activity to the parent receptor. Once ligand variants having binding activity have been identified, the binding activity of the ligand variants can be compared to each other or to a predetermined standard. Such a comparison allows identification of a ligand variant having optimal binding activity to a parent receptor.

It is understood that modifications which do not substantially affect the activity of the various embodiments of this invention are also provided within the definition of the invention provided herein. Accordingly, the following examples are intended to illustrate but not limit the present invention.

### EXAMPLE I

### Preparation of Melanophore Cells Expressing a Receptor Variant Population

This example demonstrates expression of a polypeptide receptor variant population in melanophore cells and screening ligands for binding activity.

Frog melanophore cells derived from Xenopus laevis were grown in conditioned frog media at 27° C. Conditioned frog media was made by growing frog fibroblasts in Leibovitz L-15 media (0.5× concentration) containing 20% heat inactivated fetal calf serum for 4 days, collecting the media supernatant from the fibroblasts and filtering the supernatant through a 0.2 μm filter. Frog melanophore cell cultures were periodically centrifuged through PERCOLL density gradients to enrich for more highly pigmented cells. Briefly, cells were trypsinized, suspended in quench frog media containing Leibovitz L-15 media (0.5× concentration) with 20% calf serum and centrifuged at 1500 rpm for 5 min. Cells were resuspended in 20% PERCOLL, 80% quench frog media. Cells were layered onto 2 volumes of 50% PERCOLL, 50% quench frog media and centrifuged at 600-800 rpm for 10 min. The supernatant was aspirated and cells were resuspended in quench frog media and the cells were transferred to a new tube and centrifuged at 1500 rpm for 5 min. The pellets contained melanophore cells enriched for more highly pigmented cells.

A receptor variant population is generated by identifying a region of a receptor cDNA that encodes a ligand binding site of interest. The ligand binding site of interest is excised from a parental vector using methods well known to those skilled in the art (Sambrook et al, 1989, supra). The excised fragment is used to introduce mutations in the ligand binding domain of the receptor. Mutant oligonucleotides are generated to introduce specific mutations into the ligand binding domain. Following mutagenesis, DNA corresponding to mutant ligand binding domains are introduced back into the parental vector to generate receptor variants.

Tags specific for each receptor variant also are generated. For coexpression of a receptor variant and a peptide tag, both the receptor and peptide tag are present on the parental expression vector. In parallel to excision of the ligand binding domain for mutagenesis, the DNA encoding the peptide tag is excised as well. Mutant oligonucleotides are synthesized to introduce a mutation or mutations into the receptor and simultaneously introduce a mutation or mutations into the tag. Upon introducing the mutated DNA back into the parental vector, a receptor variant is generated with a correlated tag expressed on the cell surface. Each tag is composed of specific combinations of peptides that are recognized by distinct antibodies. The antibodies are used to identify the receptor variant correlated with that tag.

Melanophore cells are transfected using electroporation (Potenza et al., Anal. Biochem. 206:315-322 (1992)). In addition, other methods well known to those skilled in the art can be used to transfect melanophores (Sambrook et al., 1989, supra). Expression of transfected proteins are assessed 2 to 3 days following transfection. Stable cell lines expressing transfected proteins can be obtained by treating cells under the appropriate selection conditions or with the appropriate drug. To minimize clonal variation, a melanophore cell line is generated that contains a chromosomally integrated neo gene for selection of neomycin resistance using G418. A loxP site is located at the 5′ end of the neo gene, but the gene has no promoter. The parental expression vector contains receptor or receptor variant DNA with its own promoter as well as a downstream promoter 3′ of the receptor DNA. LoxP sites are located at the 5′ end of the receptor DNA and at the 3′ end of the downstream promoter. The receptor or receptor variant DNA is transfected into cells and site-specific recombination occurs at the loxP sites. When site specific recombination at the loxP sites occurs, the downstream promoter is placed at the 5′ end of the neo gene, thus providing a selectable marker and an indication that site-specific recombination and introduction of the receptor or receptor variant DNA into the cells has occurred. An advantage of this loxP system is that the receptor or receptor variant is introduced into the same location in the melanophore cell genome, thus minimizing clonal variation due to different sites of integration in the genome.

Melanophore cells expressing a collective receptor variant population are plated into one or more microtiter wells. Cells are treated with one or more ligands either as individual ligands are as pools of ligand subpopulations. Ligand binding is determined by testing the effect of ligands on signaling by the receptor variants. Phototransmission at 620 nm is measured to determine those wells which are positive for ligand binding to the collective receptor population.

Following the determination of positive ligand binding, the receptor variant population can be divided into subpopulations. The subpopulations are tested for positive ligand binding. In addition, individual receptor variants can be identified using its unique coexpressed tag. Cells positive for ligand binding are segregated from non-binding receptor variants by cell sorting using the light and dark properties of the melanophores. The segregated positive cells are sequentially exposed to each antibody used to identify the peptides in each receptor variant tag for sorting cells by fluorescence activated cell sorting using a Becton Dickinson FACSort system. Cells are initially subdivided into cells that react with one or more specific antibodies before determining the unique antibody combination that identifies each individual receptor variant. The number of individual receptor variants that bind to a given ligand are determined. The receptor variants also are determined by correlating the unique tag with the mutation of specific residues in the parent receptor.

These results demonstrate the generation of a receptor variant population correlated with identifiable tags and the identification of a ligand with optimal binding activity.

### EXAMPLE II

### The Probability of Binding a Focused Library and a Diverse Library of Ligands to a Receptor

This example demonstrates the probability of binding a focused library and a diverse library of ligands to a receptor.

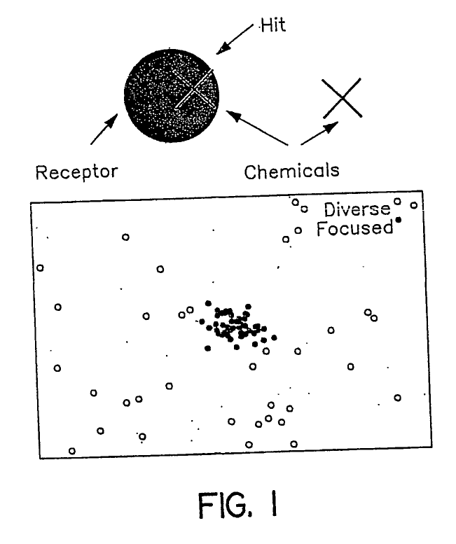

A ligand is represented as a point in space and a receptor is represented as a disc in space. A ligand binds to a receptor when the ligand lies inside the disc corresponding to the receptor (corresponding to “hit” in FIG. 1).

A ligand variant population, represented as points in space, is generated by selecting ligand variants uniformly and randomly such that the ligand variants form a distribution such as a Gaussian distribution around the parent ligand, represented as a point in space. This is accomplished by varying the chemical functional groups on the parent ligand. The closer the ligand variants fall relative to the parent ligand, the more similar the variants are chemically to the parent ligand. This is represented as the relative closeness of the points representing the ligand variants to the center of a Gaussian distribution around the point representing the parent ligand. The parameter selected to determine the Gaussian distribution of the ligand variants around the parent ligand provides a given probability of a ligand variant binding to a receptor.

Similarly, a receptor variant population, represented as discs in space, is generated by selecting receptor variants uniformly and randomly around the center of the disc in space representing the parent receptor such that the receptor variants form a distribution such as a Gaussian distribution around the parent receptor. This is accomplished by varying the chemical functional groups on the parent receptor. The closer the receptor variants fall relative to the parent receptor, the more similar the variants are chemically to the parent receptor. This is represented as the relative closeness of the points representing the receptor variants to the center of a Gaussian distribution around the center of the disc representing the parent receptor. The parameter selected to determine the Gaussian distribution of the receptor variants around the parent receptor provides a given probability that a ligand that binds to a receptor variant will also bind to the parent receptor.

The distribution of ligands and receptors is generally chosen so that the distribution of receptors is smaller than the distribution of ligands. In this case, the variance around the receptor is relatively small, reflecting receptor Variants closely related to the parent receptor. Choosing the distribution of receptors to be smaller than the distribution of ligands increases the probability that a ligand that binds to the receptor variants will also bind to the parent ligand.

In a diverse library of ligands, the ligands are distributed over a large area (see FIG. 1, bottom panel). The probability of a given ligand binding to a receptor represented as a disc in that area is decreased because there are larger gaps between the ligands. The larger gaps between ligands represent diversity of chemical functional groups of the ligands. However, there is a greater probability of binding to a larger number of receptors since the ligands are dispersed over a larger area.

In contrast to a diverse library, a focused library of ligands has ligands distributed in a smaller area due to the fact that the ligands are more closely related (see FIG. 1, bottom panel). While the probability of focused ligands binding to a variety of receptors is low due to the ligands being in a smaller area, the probability that more of the focused ligands will bind to a given receptor is high when that receptor coincides with the focused ligands. For example, if a disc representing a receptor was centered over the area covered by the focused ligands shown in FIG. 1, a number of ligands would bind to the receptor. However, the same receptor centered over the focused ligands would bind very few, if any, of the diverse ligands. Therefore, the type of ligand library is determined by the particular goals of the screen.

These results demonstrate that using a diverse library of ligands increases the probability of finding a ligand that binds to any receptor. In contrast, using a focused library of ligands increases the probability of finding a ligand that binds to a given receptor. Thus, predictions can be made as to the likelihood of identifying a ligand variant that binds to a receptor.

### EXAMPLE III

### The Probability of Identifying a Ligand that Binds a Receptor Depends on Molecular Interactions

This example demonstrates that the probability of identifying a ligand that binds a receptor depends on molecular interactions.

Binding of a ligand to a receptor generally occurs through a series of smaller interactions resulting from multiple contact points or through multiple interactions of a chemical functional group. To describe molecular interactions in a ligand-receptor binding interaction, a ligand is represented as three points in space and a receptor is represented as three discs in space. The three points representing the ligand correspond to three molecular interactions occurring through chemical groups on the ligand that serve as contact points for receptor binding. Similarly, the three discs representing the receptor correspond to three molecular interactions occurring through chemical groups on the receptor that serve as contact points for ligand binding. A ligand binds to a receptor when three points of the ligand lie inside the three discs corresponding to the receptor.

As described in Example II, parameters are selected to determine the Gaussian distribution of ligand variants around the three points representing the parent ligand. Similarly, parameters are selected to determine the Gaussian distribution of receptor variants around the three discs representing the parent receptor. In this case, the distribution around each point of the parent ligand or each disc of the parent receptor can be varied independently. For example, one point can be held to be identical to the parent molecule while the other two points are varied. Also, the distribution around the points being varied can differ from each other.

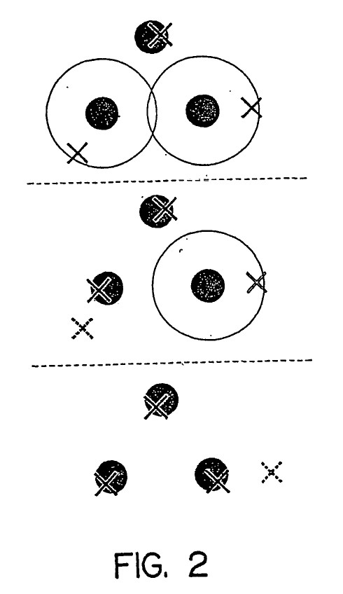

By describing a ligand-receptor binding interaction as multiple molecular interactions, an optimal binding ligand can be identified more rapidly. For example, if one of the discs representing the parent receptor is fixed to be identical to the parent receptor while the other two disc are varied to represent receptor variants, then any ligand that binds this receptor variant has an increased likelihood of binding to the parent receptor (see FIG. 2, upper panel). The increased probability of binding to the parent receptor is determined by the fact that one of the molecular interaction sites is identical to the parent. If all three discs of the receptor parent were varied, the receptor variant would be less closely related to the parent and ligands which bind to that variant have a decreased probability of binding to the parent. Fixing one molecular interaction site to be identical to the parent generates receptor variants that are more closely related to the parent. Similarly, fixing two molecular interaction sites generates receptor variants that are even more closely related to the parent receptor (see FIG. 2, middle panel).

Using a multi-point molecular interactions representation of ligand-receptor interactions provides increased probability of identifying an optimal binding ligand. For example, focused ligands can be determined in an iterative process. In a first round of screening, a receptor variant population is generated by fixing one of the three discs representing the receptor. An optimal binding ligand identified by such a screen can be used to generate a focused library of ligands. A new receptor variant population is generated by fixing two of the discs representing the receptor. This new receptor variant population is more closely related to the parent receptor. Screening the new receptor variant population with the focused library of ligands will have greatly increased probability of identifying a ligand variant with optimal binding to the parent receptor (see FIG. 2, lower panel).

These results demonstrate that considering multi-point molecular interactions in ligand-receptor binding interactions provides rapid determination of an optimal binding ligand.

### EXAMPLE IV

### The Probability of Identifying a Binding Ligand Using a Vector Representation of Ligand-Receptor Binding Interactions

This example demonstrates that a ligand and receptor binding interaction can be described as a multi-point, spatially related interaction represented as vectors.

The chemical functional groups of the ligand and the receptor are represented as vectors rather than as points and discs in space. The length of the vectors are shorter when the molecule is smaller. Therefore, smaller molecules such as organic chemicals have shorter vectors than larger molecules such as polypeptides. Each different chemical group of the ligand and receptor is represented by distinct vectors. Therefore, each ligand or ligand variant is represented by a unique string of vectors and each receptor or receptor variant is represented by a unique string of vectors.

The binding sites of a given receptor variant or ligand variant are represented by three points. The first point is the origin of the vector string. The second point is determined by starting at the origin and summing the vectors corresponding to the positions in the first half of the string. The third point is determined by starting at the second point and summing up the vectors corresponding to positions in the second half of the string. These three points define a triangle that represents each ligand or ligand variant and receptor or receptor variant. Variant molecules with similar vector strings are more closely related since they are the sum of many of the same vectors.

Binding of a ligand to a receptor is determined if the triangle representing the ligand and the triangle representing the vector can be arranged so that the points of the two triangles are close. The closeness of the triangles is measured by determining whether the lengths of the sides of the triangles representing the ligand and receptor differ by at most some threshold value. Thus, the ability of chemical groups of a ligand to bind to chemical groups of a receptor is accounted for in the vector representation as well as the spatial relationship between chemical groups of the ligand and the chemical groups of the receptor that represent binding sites.

Random noise can be introduced to represent movements of functional groups such as small changes in the relative positions of chemical groups in the molecules. In addition, random noise can be introduced to represent unknown parameters that affect ligand-receptor interactions.

To represent ligands and receptors, parameters are determined for the length of vector strings, the size of the vectors, the number of different chemical groups accounted for, the probability of a large change, the size of the random noise and the threshold for closeness of lengths of triangle sides.

The probability of finding a binding partner is determined by the variance chosen for the vectors. A high probability of finding a binding partner is provided when the vector is chosen to have small variance, which represents variants that are closely related to a parent molecule. A smaller probability of finding a binding partner is provided when the vector is chosen to have large variance, which represents variants that are more distantly related to a parent molecule. For example, when one of the binding molecules is a small molecule, the lengths of the vectors are small. If the binding partners are large molecules, the lengths of the vectors are large. Therefore, to generate a triangle with sidelengths of a similar size between large and small binding partners, a larger variance is introduced into the small molecule to increase the probability of its binding to the large molecule. In an example where a ligand is a small molecule and a receptor is a large molecule, the greatest probability of finding a binding ligand occurs when the receptor variants are closely related, represented by vectors with small variance, and the ligands are less closely related, represented by vectors with large variance. This occurs because small molecules are represented by a small number of small vectors. In order to sum this smaller number of small vectors to obtain triangle sidelengths of similar size to a large molecule, a large variance in the vectors representing the small molecule is introduced.

These results show that ligands and receptors can be represented as vectors to determine the probability of identifying a ligand that binds to a receptor.

### EXAMPLE V

### Optimization of Anti-idiotypic Antibody Ligands

This example shows that screening ligands with receptor variants increases the probability of identifying an optimal binding ligand.

The parent receptor was antibody BR96, a mouse monoclonal antibody to Le^{Y}-related cell surface antigens. Six receptor variants were generated using random codon synthesis as described in U.S. Pat. No. 5,264,563 and in Glaser et al. supra. Briefly, synthesis was performed using two DNA synthesizer columns. For simplicity, the DNA sequences are referred to as the coding strand although, in practice, all oligonucleotides were synthesized as the complementary sequence. On column 1 a trinucleotide coding for the predetermined parental codon found at the CDR positions specified below was synthesized. On column 2 a random codon encoding all 20 amino acids was synthesized using the nucleotides XXG/T where X represents a mixture of dA, dG, dC and T cyanoethyl phosphoramidites. The use of the XXG/T codon reduces the number of stop codons to include only UAG, which can be suppressed in supE E. coli bacterial strains. After synthesis of each codon, the beads from the two columns were mixed together, divided in half, and then repacked into two new columns. The columns were then returned to the DNA synthesizer and the process was repeated for the subsequent CDR positions. After the final synthesis step the contents of the two columns were pooled and the resulting oligonucleotides purified. This particular application of codon-based synthesis results in a mixture of oligonucleotides coding for randomized amino acids within a predefined region while maintaining a 50% bias toward the parental sequence at any position. By altering the proportion of the beads in the two columns, the level of substitution with respect to parental sequence can be further controlled. Furthermore, any given position can retain a specified codon and mixtures of codons other than XXG/T can be used to insert only some subset of amino acid residues if desired.

Oligonucleotides containing randomized codons were used to generate receptor variants by mutagenesis (Kunkel, Proc. Natl. Acad. Sci. USA 82:488-492 (1985) and Kunkel et al., Methods Enzymol. 154:367-382 (1987)).

Briefly, M13IXL604 or M13IXL605 phage were grown in the dut⁻ ung⁻ Escherichia coli strain CJ236 (BioRad, Richmond, Calif.) and phage were precipitated by adding 0.25 volumes of 3.5 M ammonium acetate, 20% polyethylene glycol/ml of cleared culture supernatant. Uracil-substituted single stranded DNA was isolated by phenol extraction followed by ethanol precipitation. From 6 to 8 pmol of phosphorylated oligonucleotide were used to mutagenize 250 ng of the chimeric L6 template in a 13 μl reaction volume (Huse et al., J. Immunol. 149:3914-3920 (1992).

The reaction products were diluted twofold with water and 1 μl was electroporated into E. coli strain XL-1 (Stratagene, San Diego, Calif.) and titered onto a lawn of XL-1.

Three anti-idiotypic antibody ligands were generated by immunizing 6 or 7-week-old BALB/c mice intraperitoneal (four times, once every 20 days) with 50 μg of purified antibody BR96 using aluminum hydroxide as adjuvant. The reactivity of the mice sera was tested by ELISA (Fields et al., Nature 374:739-742 (1995)). After a final boost with soluble polyclonal rabbit IgG, mice with the strongest response were killed and the spleens were used to obtain hybridomas as described (Galfre and Milstein, Methods Enzymol. 73:3-46 (1981)).

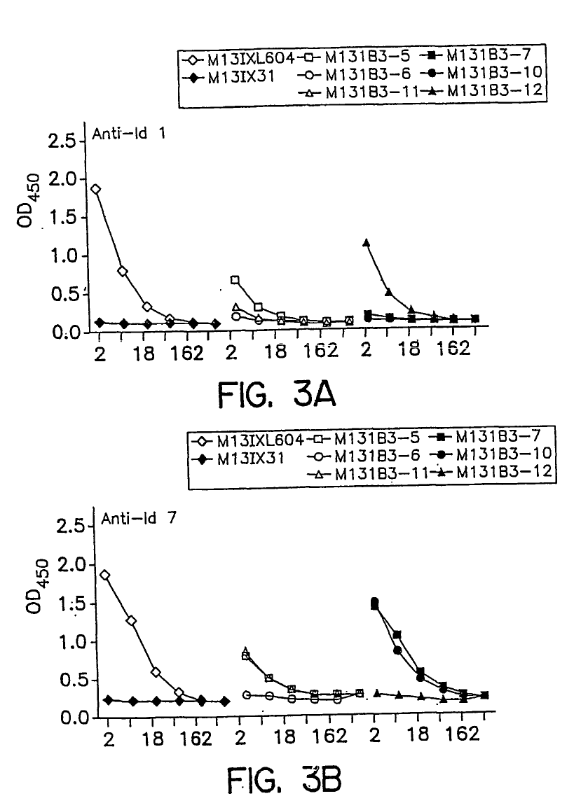

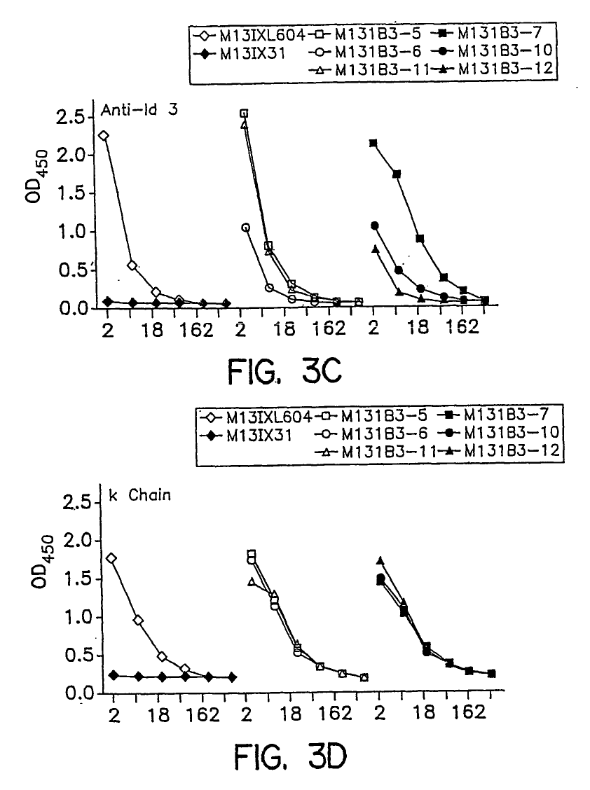

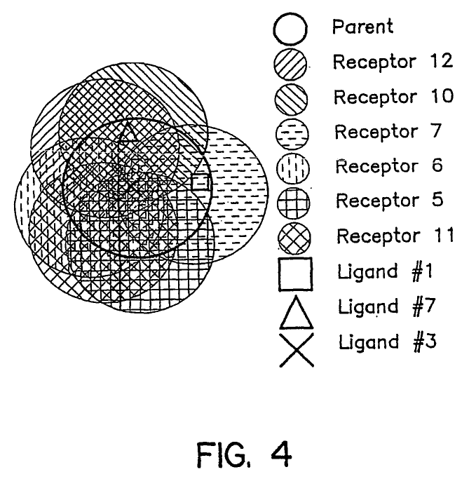

Receptor variants were screened for binding to anti-idiotypic antibody ligands. The anti-idiotypic antibody ligands were screened against the parent receptor and six receptor variants to determine binding activity using an ELISA assay (see FIG. 3). Anti-idiotypic antibody No. 1 was classified as binding to receptor 12 and the parent receptor. Anti-idiotypic antibody No. 7 was classified as binding to receptor 7, receptor 10 and the parent receptor. Anti-idiotypic antibody No. 3 was classified as binding to all of the receptors, including the parent receptor.

The nucleotide and amino acid sequences of the light chain CDR regions 1 and 2 of the parent receptor (designated wild type) and the six receptor variants (designated M131B3-5 through M131B3-12) are shown in Table 1. The nucleotide and amino acid sequences (SEQ ID NOS: 1, 3, 5, 7, 9, 11, 13, and 2, 4, 6, 8, 10, 12, 14, respectively) for the CDR L1 region of the parent and six receptor variants are shown in the top half of Table 1. The nucleotide and amino acid sequence (SEQ ID NOS: 15, 17, 19, 21, 23, 25, 27 and 16, 18, 20, 22, 24, 26, 28, respectively) for the CDR L2 region of the parent and six receptor variants are shown in the bottom half of Table 1. In Table 1, L1 and L2 CDR mutations in M13IXL604 clones were selected on the basis of binding to anti-idiotypic antibody No. 3 similar to that of wild type and negligible binding to anti-idiotypic antibody No. 1. Changes resulting from the mutagenesis procedure are indicated by boldface type.

Several positions in the receptor sequence were found to be conserved while other positions were found to differ from the parent receptor in both CDR regions 1 and 2. Substitutions occurred at all five target loci in CDR L1 and at three loci in CDR L2. The total number of substitutions in CDR L1 and CDR L2 ranged from two to four in each mutant.

TABLE 1

Nucleotide and Amino Acid Sequences of Receptor
Variants of ER96 Antibody

| Amino Acid | 26 27 28 29 30 31 32 33 |
|---|---|
| CDR L1 | |
| Wild type | AGC TCA AGT GTA AGT TTC ATG AAC |
| | Ser Ser Ser Val Ser Phe Met Asn |
| M131B3-5 | AGC TCA AGT GTA AGG TTC ATG AAC |
| | Ser Ser Ser Val Arg Phe Met Asn |
| M131B3-6 | AGC GAG AGT GTA AAT CTT ATG AAC |
| | Ser Glu Ser Val Asn Leu Met Asn |
| M131B3-7 | AGC TCA AGT GTT AAT TTC ATG AAC |
| | Ser Ser Ser Val Asn Phe Met Asn |
| M131B3-10 | AGC TCA ACG GTA AGT TTC ATG AAC |
| | Ser Ser Thr Val Ser Phe Met Asn |
| M131B3-11 | AGC TCA AGT GTA GCG TAT ATG AAC |
| | Ser Ser Ser Val Ala Tyr Met Asn |
| M131B3-12 | AGC CAG AGT GCT AAG CAT ATG AAC |
| | Ser Gln Ser Ala Lys His Met Asn |
| Amino Acid | 49 50 51 52 53 54 55 56 |
| CDR L2 | |
| Wild type | GCC ACA TCC AAT TTG GCT TCT GGA |
| | Ala Thr Ser Asn Leu Ala Ser Gly |
| M131B3-5 | GCC ACA GAG AAG TTG GCT TCT GGA |
| | Ala Thr Glu Lys Leu Ala Ser Gly |
| M131B3-6 | GCC ACA GTT AAT TTG GCT TCT GGA |
| | Ala Thr Val Asn Leu Ala Ser Gly |
| M131B3-7 | GCC ACA GTG AAT TTG GCT TCT GGA |
| | Ala Thr Val Asn Leu Ala Ser Gly |
| M131B3-10 | GCC ACA TCC AGG GCG GCT TCT GGA |
| | Ala Thr Ser Arg Ala Ala Ser Gly |
| M131B3-11 | GCC ACA CAG AAT TTG GCT TCT GGA |
| | Ala Thr Gln Asn Leu Ala Ser Gly |
| M131B3-12 | GCC ACA TCC AAT TTG GCT TCT GGA |
| | Ala Thr Ser Asn Leu Ala Ser Gly |

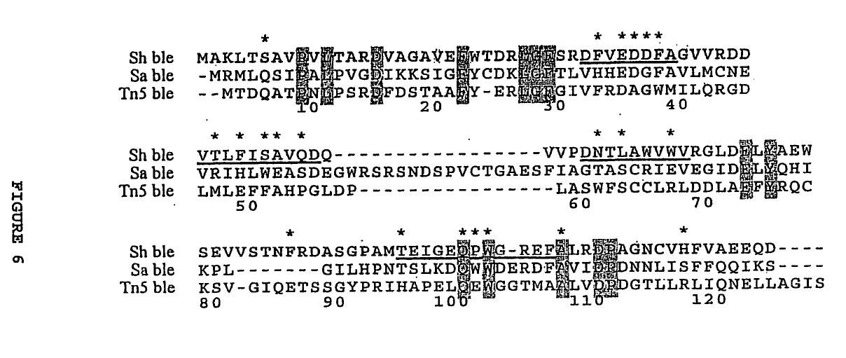

The results of the screen are summarized in FIG. 6, where receptors are represented as discs and ligands are represented as symbols. These results demonstrate that screening ligands against a population of receptor variants will rapidly identify ligands having optimal binding activity. For example, if the collective receptor variant population of this example were screened in the melanophore system, ligand No. 3 would have generated the highest signal since it binds to all seven receptors in the receptor variant population. Ligand No. 7 would give a weaker signal since this ligand binds to three receptors in the receptor variant population. Ligand No. 1 would give a still weaker signal since this ligand binds to two receptors in the receptor variant population. Thus, screening with a collective receptor variant population provides more information about the binding characteristics of the ligand than screening with the parent receptor alone. In addition, ligands that bind weakly to the parent receptor may not have been detectable above background when screened against the parent alone but are detectable when more than one receptor in the receptor variant population binds to the ligand.

These results demonstrate that screening a receptor variant population rapidly identifies optimal binding ligands to a receptor.

### EXAMPLE VI

### Modification of the Doublelox Targeting Vector

This example describes modification of the doublelox targeting vector.

The doublelox targeting vector pBS397-p53cat could not be used as a general vehicle for applying directed evolution technologies to a wide range of proteins because the synthetic polylinker region contained a limited number of unique restriction sites that hindered rapid cloning of the target protein(s) of interest. Moreover, the vector did not contain the filamentous phage origin of replication and, consequently, could not be used to generate single-stranded DNA template for oligonucleotide-directed mutagenesis. Therefore, to facilitate the future synthesis of libraries of variants of BRP and other target proteins, the f1 origin of replication was cloned into the doublelox targeting vector.

DNA encoding the f1 origin was obtained by treating pcDNA3.1/Zeo (Invitrogen; Carlsbad, Calif.) with SphI restriction endonuclease to generate a 575 base pair fragment containing the f1 origin, and the pBS397 doublelox targeting vector was treated with SfI1 restiction endonuclease. Both the f1 origin-containing fragment and the linearized pBS397 were treated with T4 polymerase to create blunt ends, and the fragment was ligated with the vector. To select for the proper orientation, the ligated vector was treated with two restriction endonucleases, one with a unique site within the f1 origin (XhoI) and the other with a unique site within the vector (DraIII).

Modified pBS397 vector containing the f1 origin in the (+) orientation, termed pBS397-f1(+), was selected based on the size of the fragment generated following treatment with XhoI and DraIII and subsequently was characterized more fully by DNA sequencing. Because the modified doublelox targeting vector contains the filamentous phage f1 origin of replication, single-stranded uracil-containing DNA template of BRP or any other target protein of interest can be routinely obtained and used to synthesize libraries of protein variants based on oligonucleotide-directed mutagenesis.

The filamentous phage f1 origin of replication was cloned into the doublelox targeting vector. This permitted the efficient and precise synthesis of protein libraries by oligonucleotide-directed mutagenesis.

### EXAMPLE VII

### Cloning of BRP and Expression of BRP in NIH3T3 Cells

This example describes cloning of BRP into the targeting vector pBS397-f1(+) and expression of BRP in the mammalian NIH3T3 target call line 13-1.

To clone BRP into the targeting vector, a DNA fragment containing the CMV (eukaryotic) and EM7 (bacterial) promoters, the BRP gene product, and the SV40 polyadenylation sequence was removed from the pCMV/Zeo vector (Invitrogen; Carlsbad, Calif.) by treatment with restriction endonucleases EcoRV and HindIII. Likewise, the modified doublelox targeting vector pBS397-f1(+) was also treated with endonucleases EcoRV and HindIII. Subsequently, the insert containing BRP gene product was ligated with the linearized vector to yield a new vector (pBS397-f1(+)/BRP) containing the CMV and EM7 promoters, BRP gene product, the SV40 polyadenylation sequence, and the 3′ terminal portion of the neo gene all flanked by the doublelox sites.

To express BRP in mammalian cells, the host mammalian cell line 13-1, which was derived from mouse NIH3T3 cells and contains a single copy of lacZ reporter gene flanked by heterospecific loxP sites oriented head-to-tail, was used (FIG. 5C) (Bethke and Sauer, Nuc. Acids Res., 25:2828-2834 (1997)).

The host cell line also contains an ATG start and promoter for neo gene expression and a functional lacZ gene, resulting in a G418-sensitive/blue phenotype. The doublelox targeting vector contains a disabled neo gene and BRP flanked by heterospecific loxP sites (FIG. 5C) with an expression STOP signal upstream of the heterospecific lox sites to diminish illegitimate expression events (Sauer, Methods Enzymol. 225:890-900 (1993)). Site-specific recombination by the doublelox targeting vector resulted in excision of the lacZ gene and expression of the neo gene, generating a G418-resistant/white phenotype.

The sensitivity of the host NIH3T3 target cell line 13-1 to the antibiotic Zeocin was determined. Zeocin, a glycopeptide member of the bleomycin/phleomycin family of antibiotics, is found in Streptomyces verticillus and displays strong toxicity against bacteria, fungi, plants, and mammalian cell lines (Drocourt et al., Nucleic Acids Res., 18:4009 (1990); Calmels et al., Curr. Genet. 20:309-314 (1991); Perez et al., Plant Mol. Biol., 13:365-373 (1989); Mulsant et al., Somat. Cell Mol. Genet., 14:243-252 (1988)). The toxicity of Zeocin arises from its ability to intercalate into and cleave DNA. However, Zeocin resistance due to stoichiometric binding and inactivation by the Sh Ble gene product (BRP) has been observed and, consequently, BRP has been used as a selectable marker to confer resistance to Zeocin in both prokaryotes and eukaryotes.

Mammalian cells exhibit a wide range of susceptibilities to Zeocin, which is influenced by the cell line and other factors such as ionic strength, cell density, and growth rate. Consequently, prior to expressing and screening libraries of BRP variants, the sensitivity of the NIH3T3-derived 13-1 host cell line to Zeocin was determined. To determine the Zeocin sensitivity, the 13-1 cells were plated at approximately 25% confluency. Twenty-four hours later, the media was replaced with fresh media containing 0, 50, 100, 200, 400, 800, or 1000 μg/ml Zeocin. The selective media was replaced every 4 days, and the percentage of surviving cells was examined over 14 days. As reported by the manufacturer (Invitrogen), the response of cells to Zeocin was distinct from other selectable agents such as neomycin that cause susceptible cells to round up and detach from the plate. Cells susceptible to Zeocin treatment exhibited abnormal shapes and large increases in size. Large empty cytoplasmic vesicles were observed at higher magnifications. Treatment of the host 13-1 cell monolayers with ≧100 μg/ml Zeocin killed the cells, indicating that the host cell line was sensitive to treatment with 100 μg/ml Zeocin, though the toxicity was evident sooner at Zeocin concentration ≧400 μg/ml. Essentially all cells were killed in 7-10 days in ≧400 μg/ml Zeocin. The Zeocin sensitivity of the 13-1 host cell line is consistent with previous observations that most mammalian cell lines are susceptible to Zeocin at concentrations ranging from 50-1000 μg/ml in selective medium.

To determine Zeocin sensitivity of the host cell line 13-1 transfected with BRP, the host cell line 13-1 was co-transfected with the pBS397-f1(+)/BRP doublelox targeting vector and the pBS185 Cre recombinase vector using the conditions described previously (Bethke and Sauer, Nuc. Acids Res., 25:2828-2834 (1997)). Briefly, 5×10⁵ host 13-1 cells were transfected overnight in a 100-mm dish with 4 μg pBS185 and 30 μg pBS397-f1(+)/BRP using calcium phosphate (Chen and Okayama, Mol. Cell. Biol., 7:2745-2752 (1987)). Transformants arising from Cre-mediated targeted insertion were selected 48 hours later by replating in media containing 400 μg/ml geneticin. Colonies were isolated and transferred to 24-well culture plates 10 days later. As described previously, targeted insertion with the doublelox vector resulted in excision of lacZ and expression of the neo and Sh ble gene products. Stable clones expressing BRP were further confirmed by PCR.

Using the Zeocin selection protocol described above, the resistance of 13-1 host cells transformed with BRP was determined. Zeocin concentrations ranging from 50-1000 μg/ml did not kill or inhibit the proliferation of the transformed cells. Control cells transfected with unmodified doublelox targeting vector not expressing the BRP gene displayed sensitivity to Zeocin similar to the untransformed host cells. Specifically, the control cells were sensitive to treatment with ≧100 μg/ml Zeocin. The mechanism of BRP inactivation of Zeocin is sequestration through binding and, consequently, is stoichiometric. Therefore, to determine if the Zeocin resistance introduced by BRP transformation of the cells could be overcome, the cells were treated with higher concentrations of Zeocin (2500 and 5000 μg/ml). The cells transformed with BRP were resistant to 2500 μg/ml Zeocin but were killed by treatment with 5000 μg/ml Zeocin, consistent with the BRP binding sites being saturated.

The Zeocin sensitivity of multiple distinct clones of the host cell line stably transfected with BRP using the targeted integration was characterized. Importantly, all of these clones displayed similar Zeocin sensitivity profiles in which the cells were resistant to treatment with 2500 μg/ml Zeocin but killed by treatment with 5000 μg/ml Zeocin. Because Zeocin resistance depends on the stoichiometric binding of Zeocin by BRP, data indicate that the different clones express similar levels of the BRP protein. Subsequently, Western blot analysis demonstrated that BRP protein expression levels were similar in different clones. The relatively uniform protein expression levels observed support the advantageous use of the recombination system, resulting in every BRP transformant expressing the gene at the same genomic location.

These results indicate that transformation of the host target cell line with BRP resulted in sensitivity of the transformants to Zeocin. Multiple distinct clones were found to express similar amounts of BRP.

### EXAMPLE VIII

### Optimization of Transfection Parameters for Site-Specific Integration

This example describes optimizing transfection parameters for Cre-mediated site-specific integration of BRP in 13-1 cells for expressing libraries of BRP variants.

Calcium phosphate transfection of 13-1 cells was previously demonstrated to result in targeted integration in 1% of the viable cells plated (Bethke and Sauer, Nuc. Acids Res., 25:2828-2834 (1997)). Therefore, initial studies were conducted using calcium phosphate to transfect 13-1 cells with 4 μg pBS185 and 10, 20, 30, or 40 μg of pBS397-f1(+)/BRP. The total level of DNA per transfection was held constant using unrelated pBluescript II KS DNA (Stratagene; La Jolla, Calif.), and transformants were selected 48 hours later by replating in media containing 400 μg/ml geneticin. Colonies were counted 10 days later to determine the efficiency of targeted integration. Optimal targeted integration was typically observed using 30 μg of targeting vector and 4 μg of Cre recombinase vector pBS185, consistent with the 20 μg targeting vector and 5 μg of pBS185 previously reported (Bethke and Sauer, Nuc. Acids Res., 25:2828-2834 (1997)). The frequency of targeted integration observed was generally <1%. The observed variability was due, in part, to the fastidious nature of the calcium phosphate methodology. For example, the methodology was particularly sensitive to the amount of DNA used and the buffer pH, and both parameters displayed a narrow optimum range, although targeted integration efficiencies observed were sufficient to express the protein libraries.

Other transfection methods were also characterized. In general, lipid-mediated transfection methods are more efficient than methods that alter the chemical environment, such as calcium phosphate and DEAE-dextran transfection. In addition, lipid-mediated transfections are less affected by contaminants in the DNA preparations, salt concentration, and pH and thus generally provide more reproducible results (Felgner et al., Proc. Natl. Acad. Sci. USA, 84:7413-7417 (1987)).

Consequently, a formulation of the neutral lipid dioleoyl phosphatidylethanolamine and a cationic lipid, termed GenePORTER transfection reagent (Gene Therapy Systems; San Diego, Calif.), was evaluated as an alternative transfection approach. Briefly, endotoxin-free DNA was prepared for both the targeting vector pBS397-f1(+)/BRP and the Cre recombinase vector p-BS185 using the EndoFree Plasmid Maxi kit (QIAGEN; Valencia, Calif.). Next, 5 μg pBS185 and varying amounts of pBS397-f1(+)/BRP were diluted in serum-free medium and mixed with the GenePORTER transfection reagent. The DNA/lipid mixture was then added to a 60-70% confluent monolayer of 13-1 cells consisting of approximately 5×10⁵ cells/100-mm dish and incubated at 37° C. Five hours later, fetal calf serum was added to 10%, and the next day the transfection media was removed and replaced with fresh media.

Transfection of the cells with variable quantities of the targeting vector yielded targeted integration efficiencies ranging from 0.1% to 1.0%, with the optimal targeted integration efficiency observed using 5 μg each of the targeting vector and the Cre recombinase vector. Lipid-based transfection of the 13-1 host cells under the optimized conditions resulted in 0.5% targeted integration efficiency being consistently observed. Although 0.5% targeted integration is slightly less than the previously reported 1.0% efficiency (Bethke and Sauer, Nuc. Acids Res., 25:2828-2834 (1997)), it is sufficient to express large protein libraries and allows expressing libraries of protein variants in mammalian cells.

These results demonstrate optimization of transfection conditions for targeted insertion in NIH3T3 13-1 cells. Conditions for a simple, lipid-based transfection method that required a small amount of DNA and generated reproducible 0.5% targeting efficiency were established.

### EXAMPLE IX

### Synthesis of Focused BRP Libraries by Codon-Based Mutagenesis

This example describes the synthesis of focused BRP libraries directed to specific regions of BRP using codon-based mutagenesis.

In vivo, molecular evolution is likely to proceed through the step-wise accumulation of discreet mutations that do not diminish function. Therefore, to mimic this process in vitro, focused libraries consisting of BRP variants containing a single amino acid change were synthesized and expressed using codon-based mutagenesis (Glaser et al., J. Immunol., 149:3903-3913 (1992)). Based on site-directed mutagenesis studies and structural modeling of BRP and related proteins, certain residues located predominantly within four distinct regions of the BRP linear sequence were predicted to be involved in bleomycin binding (FIG. 6) (Dumas et al., EMBO J. 13:2483-2492 (1994)). Therefore, every position in all four of the binding regions underlined in FIG. 6 was mutated, one at a time, resulting in the subsequent expression of all 20 amino acids at each residue of the binding region.

A summary of the four BRP libraries consisting of variants that each contains a single amino acid mutation is shown in Table 2. The libraries created through this approach ranged in size from 256 (region 1) to 412 (region 4) unique members and contained a total of 1,280 BRP variants. The libraries were focused and therefore were considerably smaller than those that would be obtained through total randomization. For example, while application of codon-based mutagenesis to BRP region 1 (residues 32-39) resulted in a library containing 160 unique protein variants, complete randomization of the same region would yield >10¹⁰ unique clones, of which only a minor fraction would display the desired function.

Several advantages were expected to be derived from utilizing smaller libraries that introduce incremental structural changes. First, a greater proportion of the BRP library should be functional because the binding activity will not have been destroyed by extensive mutagenesis. Next, the lower complexity of the libraries should result in the identification of variants with modified affinity at a higher frequency than achievable in completely randomized libraries. As a result, assays more predictive of function can be used. Finally, because the libraries are smaller and easily screened, the contribution of all four binding regions to bleomycin (Zeocin) binding can be assessed.

A summary of the BRP libraries generated is shown in Table 2. The location is based on the amino acid numbering depicted in FIG. 6. The length refers to the number of amino acids included at each library site, and the library diversity reflects the maximum potential DNA diversity based on using NN(G/T) codons for mutagenesis.

TABLE 2
Summary of BRP Libraries.

| Library Site | Location | Length | Library Diversity |
|---|---|---|---|
| 1 | 32-39 | 8 | 256 |
| 2 | 46-55 | 10 | 320 |
| 3 | 60-68 | 9 | 288 |
| 4 | 95-107 | 13 | 416 |

The oligonucleotides encoding the variants containing a single amino acid mutation were cloned into the doublelox targeting vector using oligonucleotide-directed (hybridization) mutagenesis (Kunkel, Proc. Natl. Acad. Sci. USA, 82:488-492 (1985)). In order to characterize the quality of the libraries and the efficiency of mutagenesis, the DNA from approximately 15-20 randomly selected transformants from each library was sequenced (Table 3).

The efficiency of mutagenesis of BRP, defined as the percentage of clones containing mutations, ranged from 56% (library 4) to 75% (library 1). Single amino acid changes were distributed across each library region, and multiple distinct amino acid changes were identified at single sites. For example, characterization of as few as 16 randomly selected clones from library 1 identified mutations at 7 of 8 positions (distribution of mutations across a library region) and provided an example of three mutations at position Phe34 (multiple distinct amino acids at a single site). Further evidence of the diversity of the BRP libraries was provided by the low frequency at which identical clones were randomly selected. Cumulatively, in sequencing 70 randomly selected clones, only five variants were identified more than once (clones 1.5, 2.1, 2.8, 3.1, and 4.4 were identified twice each).

Library characterization using DNA sequencing revealed an error that was made during the synthesis of the mutagenic oligonucleotides. Specifically, during oligonucleotide synthesis, the wild type Ala65 was inadvertently changed to Gly65. Consequently, the majority of variants arising from the oligonucleotide pool that was intended to encode single amino acid changes actually contained two mutations. Despite the inadvertent mutation, library 3 was screened for BRP activity because the principal objective of this study was to demonstrate efficient expression of protein libraries in mammalian cells, and the actual composition of the library was not expected to affect the efficiency of Cre-mediated targeted insertion. Moreover, although the majority of clones from this library contained two mutations, Ala65 is not conserved in the family of gene products (FIG. 6) and has not previously been identified as critical for function. Thus, despite containing two mutations, the variants are still closely related to the wild type BRP. Finally, the ⁶⁵Ala to Gly mutation is a conserved substitution and was not expected to introduce substantial structural changes.

Table 3 shows a summary of the amino acid sequences of randomly selected BRP variants (Library 1, SEQ ID NOS:34-44; Library 2, SEQ ID NOS:45-54; Library 3, SEQ ID NOS:55-65; Library 4, SEQ ID NOS:66-73). Clones with silent mutations (2.10, 2.11, 4.8, and 4.9) contained altered DNA sequence consistent with oligonucleotide-directed mutagenesis. However, the altered DNA sequence encoded the same amino acid encoded by wild type BRP DNA.

TABLE 3
Summary of amino acid sequence or randomly
selected BRP variants.

| Li- | # | Desig- | | | | | | | | | | | | | | |
|---|---|---|---|---|---|---|---|---|---|---|---|---|---|---|---|---|
| brary | Sequenced | nation | n | Sequence | | | | | | | | | | | | |
| 1 | 16 | WT | 4 | D | F | V | E | D | D | F | A | | | | | |
| | | 1.1 | 1 | R | | | | | | | | | | | | |
| | | 1.2 | 1 | | L | | | | | | | | | | | |
| | | 1.3 | 1 | | S | | | | | | | | | | | |
| | | 1.4 | 1 | | G | | | | | | | | | | | |
| | | 1.5 | 2 | | | C | | | | | | | | | | |
| | | 1.6 | 1 | | | | Y | | | | | | | | | |
| | | 1.7 | 1 | | | | | L | | | | | | | | |
| | | 1.8 | 1 | | | | | G | | | | | | | | |
| | | 1.9 | 1 | | | | | | | S | | | | | | |
| | | 1.10 | 1 | | | | | | | | R | | | | | |
| | | 1.11 | 1 | (deletion) | | | | | | | | | | | | |
| 2 | 18 | WT | 5 | V | T | L | F | I | S | A | V | Q | D | | | |
| | | 2.1 | 2 | L | | | | | | | | | | | | |
| | | 2.2 | 1 | A | | | | | | | | | | | | |
| | | 2.3 | 1 | | | | L | | | | | | | | | |
| | | 2.4 | 1 | | | | | V | | | | | | | | |
| | | 2.5 | 1 | | | | | | N | | | | | | | |
| | | 2.6 | 1 | | | | | | I | | | | | | | |
| | | 2.7 | 1 | | | | | | | | | T | | | | |
| | | 2.8 | 2 | | | | | | | | | H | | | | |
| | | 2.9 | 1 | | | | | | | | | | P | | | |
| | | 2.10-11 | 1 | (silent mutations) | | | | | | | | | | | | |
| 3 | 18 | WT | 7 | D | N | T | L | A | W | V | W | V | | | | |
| | | 3.1 | 2 | | D | | G | | | | | | | | | |
| | | 3.2 | 1 | | L | | G | | | | | | | | | |
| | | 3.3 | 1 | | | P | | G | | | | | | | | |
| | | 3.4 | 1 | | | | M | G | | | | | | | | |
| | | 3.5 | 1 | | | | | C | | | | | | | | |
| | | 3.6 | 1 | | | | | S | | | | | | | | |
| | | 3.7 | 1 | | | | | G | | W | | | | | | |
| | | 3.8 | 1 | | | | | G | | | R | | | | | |
| | | 3.9 | 1 | | | | | G | | | | L | | | | |
| | | 3.10 | 1 | | | | | | | | | C | | | | |
| 4 | 18 | WT | 8 | T | E | I | G | E | Q | P | W | G | R | E | F | A |
| | | 4.1 | 1 | V | | | | | | | | | | | | |
| | | 4.2 | 1 | | S | | | | | | | | | | | |
| | | 4.3 | 1 | | | | | W | | | | | | | | |
| | | 4.4 | 2 | | | | | | H | | | | | | | |
| | | 4.5 | 1 | | | | | | | | L | | | | | |
| | | 4.6 | 1 | | | | | | | | | | | | G | |
| | | 4.7 | 1 | | | | | | | | | | | | | S |

| | | |
|---|---|---|
| 4.8-9 | 1 | (silent mutations) |

These results describe the generation of focused BRP libraries. Hybridization mutagenesis of BRP using oligonucleotides synthesized by codon-based mutagenesis introduced the desired diversity focused across the regions of interest.

### EXAMPLE X

### Functional Screening of BRP Libraries Expressed In Mammalian Cells

This example describes functional screening of BRP libraries expressed in mammalian cells.

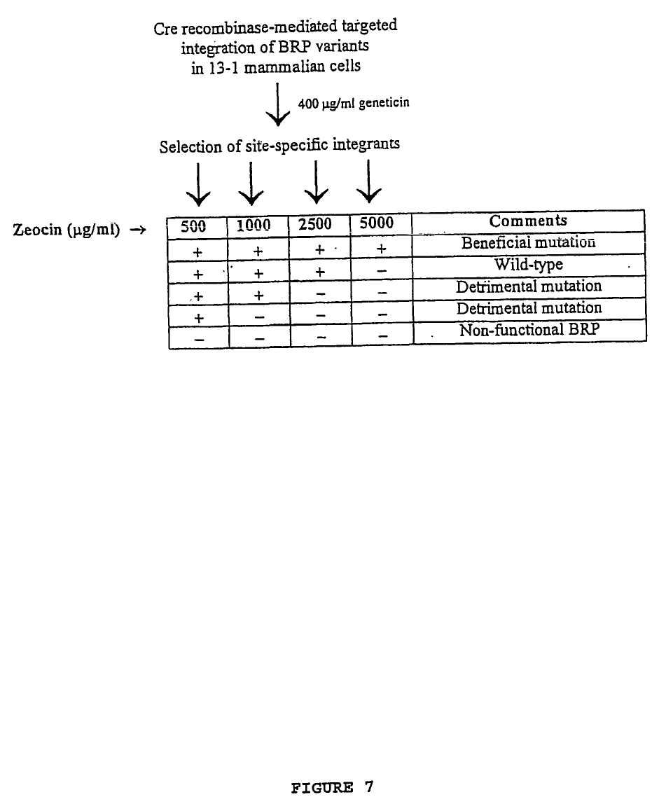

Each of the four BRP libraries was used to transform the mammalian host cell line 13-1 using optimized conditions described in Example VIII, and site-specific integrants were selected with geneticin. Host cells transformed with BRP variants were identified based on resistance to geneticin and subsequently were isolated, expanded, and screened for Zeocin sensitivity (FIG. 7). After proliferation to obtain a sufficient number of cells, each clone was plated in four separate wells to permit exposure to variable concentrations of Zeocin for 14 days. Similar to previous results, clones transformed with wild type BRP were resistant to 500, 1000, and 2500 μg/ml Zeocin but were killed by treatment with 5000 μg/ml Zeocin. Therefore, in order to identify BRP variants with beneficial mutations conferring increased affinity for Zeocin, one sample of all clones was treated with 5000 μg/ml Zeocin. Conversely, to identify mutations that diminished binding to Zeocin, that is, sensitive to 2500 μg/ml Zeocin, cultures of each clone were treated with 500 or 1000 μg/ml Zeocin. Clones that were sensitive to 500 μg/ml Zeocin were not characterized further but presumably include mutations that render BRP non-functional due to disruption of critical binding residues or substantial perturbation of the structure of BRP.

Site-specific targeted integrants were selected by placing the transfected cells in media containing geneticin. Following the outgrowth of colonies, separate cultures of each clone were grown in the presence of the indicated concentration of Zeocin. The phenotypes of the BRP variants were categorized as beneficial (resistant to 5000 μg/ml Zeocin), wild type (resistant to 2500 μg/ml Zeocin), detrimental (resistant to 500 and 1000 μg/ml Zeocin), or non-functional (sensitive to 500 μg/ml Zeocin). The variants were categorized as shown in FIG. 7.

Treatment of the clones transformed with BRP mutants with varying amounts of Zeocin led to the identification of multiple clones displaying altered sensitivities to Zeocin, with detrimental mutations being identified most frequently. The predominance of detrimental mutations following Zeocin selection is consistent with previous directed evolution studies performed with unrelated proteins (Wu et al., Proc. Natl. Acad. Sci. USA, 95:6037-6042 (1998); Wu et al., J. Mol. Biol., 294:151-162 (1999), and undoubtedly reflects the efficiency of molecular evolution in vivo. Moreover, the multiple examples of impaired BRP function arising from altering BRP by a single amino acid underscores the advantages of using a focused mutagenesis strategy for applying directed evolution approaches.

Clones displaying the wild type phenotype (resistant to 2500 μg/ml Zeocin) were not analyzed further in the present studies because characterization of the libraries by DNA sequencing demonstrated that 25-54% of the clones expressed wild type BRP (Table 3). To identify the precise location and nature of the mutations, the DNA encoding the BRP variants was sequenced. Briefly, total cellular DNA was isolated from approximately 10⁴ cells of each clone of interest using DNeasy Tissue Kits (QIAGEN; Valencia, Calif.). Next, the BRP gene contained within the complex genomic DNA was amplified using PfuTurbo DNA polymerase (Stratagene; La Jolla, Calif.), an enhanced version of Pfu DNA polymerase used for high fidelity PCR, and oligonucleotide primers that flanked the Sh ble gene (BRP). An aliquot of the PCR product was then used to sequence BRP by the fluorescent dideoxynucleotide termination method (Perkin-Elmer) using a nested oligonucleotide primer.

DNA sequencing demonstrated that the clones displaying differential sensitivity to Zeocin contained a variety of mutations (Table 4)(Library 1, SEQ ID NOS:34, 74-77, 36 and 78, respectively; Library 2, SEQ ID NOS:45, 46 and 79-81, respectively; Library 3, SEQ ID NOS:55 and 82-85, respectively; Library 4, SEQ ID NOS:66 and 86-88, respectively). Mutations of residues predicted to be involved in bleomycin binding (Dumas et al., EMBO J. 13:2483-2492 (1994)) were mostly detrimental as demonstrated by enhanced sensitivity to Zeocin (clones 1E, 2C, 3A-D, for example). A notable exception was clone 1B, in which the mutation of ³⁸Asp to Asn resulted in increased resistance to Zeocin. However, mutation of Asn to Asp for solvent exposed residues is not an uncommon substitution from a protein evolutionary perspective.

TABLE 4
Summary of select BRP Variants.

| | | | | | | | | | | | | | | | Zeocin |
|---|---|---|---|---|---|---|---|---|---|---|---|---|---|---|---|
| Library | Clone | Sequence | | | | | | | | | | | | | Resistance |
| 1 | WT | D | F | V | E | D | D | F | A | | | | | | 2500 |
| | 1A | | | Y | | | | | | | | | | | 500 |
| | 1B | | | | | | N | | | | | | | | 5000 |
| | 1C | F | | | | | | | | | | | | | 5000 |
| | 1D | C | | | | | | | | | | | | | 2500 |
| | 1E | | L | | | | | | | | | | | | 1000 |
| | 1F | G | | | | | | | | | | | | | 1000 |
| 2 | WT | V | T | L | F | I | S | A | V | Q | D | | | | 2500 |
| | 2A | L | | | | | | | | | | | | | 5000 |
| | 2B | | I | | | | | | | | | | | | 2500 |
| | 2C | | | | | | | T | | | | | | | 1000 |
| | 2D | | | | | | | | L | | | | | | 5000 |
| 3 | WT | D | N | T | L | A | W | V | W | V | | | | | 2500 |
| | 3A | | | | | | | | L | | | | | | 500 |
| | 3B | | | | S | G | | | | | | | | | 1000 |
| | 3C | | | | | G | | | L | | | | | | 500 |
| | 3D | | | | | G | | | C | | | | | | 500 |
| 4 | WT | T | E | I | G | E | Q | P | W | G | R | E | F | A | 2500 |
| | 4A | | P | | | | | | | | | | | | 1000 |
| | 4B | | | L | | | | | | | | | | | 500 |
| | 4C | | | | | S | | | | | | | | | 1000 |

Shuffling of DNA from families of genes has been-used to generate diversity for the creation of protein libraries for directed evolution and has resulted 10 in the identification of protein variants with improved function (Crameri et al., Nature, 391:288-291 (1998); Chang et al., Nature Biotech. 17:793-797 (1999)). In the present study, three clones with altered phenotypes contained mutations to amino acids found in related proteins. For example, the ⁴⁷Val to Leu (clone 2A) and the ⁹⁸Ile to Leu (clone 4B) mutations convert the amino acids to those expressed in the Th5 ble and Sa ble gene products, respectively. Clone 3B, which unintentionally contained both ⁶⁴Leu to Ser and ⁶⁵Ala to Gly, displayed increased Zeocin sensitivity despite the fact that both the Th5 ble and Sa ble gene products express Ser at residue 64. However, a mutant containing only the ⁶⁵Ala to Gly mutation displayed even greater sensitivity to Zeocin, suggesting that the 64Leu to Ser mutation might be compensatory for ⁶⁵Ala to Gly. Thus, precise and thorough mutagenesis of defined regions of BRP identified beneficial mutations that would have arisen from DNA shuffling techniques.

Within the four regions of BRP selected for the synthesis of focused libraries, only residues Gln102, Trp104, and Ala109, all located in region four, are conserved among all three related gene products. No functional BERP variants with mutations in any of these three positions were identified following Zeocin selection. The trivial explanation that mutations at these particular residues occurred at low frequency in the library was ruled out based on the DNA sequencing of clones randomly selected from library 4 (Table 3). One mutation at each of these three sites was identified even though only 18 clones in total were characterized. The inability to identify functional variants with mutations at residues Gln102, Trp104, and Ala109 is consistent with the finding that these residues are conserved in all members of the gene family.

Clone 2D displays enhanced resistance to Zeocin resulting from a conserved ⁵⁴Val to Leu mutation that illustrates the benefits of directed evolution approaches to protein engineering. Each member of the gene family expresses a distinct residue at position 54, and previous predictions based on structural modeling and site-directed mutagenesis have not identified Va154 as a potentially important residue. Consequently, in addition to validating structural predictions, application of directed evolution technologies identified new mutations, providing additional structural information indirectly.

Libraries of proteins occasionally contain clones expressing unintentional mutations, introduced either through minor impurities in the oligonucleotides used for mutagenesis or by random mutagenesis in vivo following transformation. Typically, these mutations occur at low frequencies that do not impact the success of screening and are not detected by characterization of the libraries by DNA sequencing. Nonetheless, to verify that altered function of a clone of interest is not a result of additional mutations at other sites in the protein, the entire DNA sequence of clones of interest was determined. For example, in the present study, DNA sequencing of clone 3A demonstrated that it contains two mutations, ⁶⁵Gly to Ala and ⁶⁸Trp to Leu. The ⁶⁵Gly to Ala mutation was not immediately obvious because it “corrected” the mutation originally introduced as a mistake during the synthesis of mutagenesis oligonucleotides. Despite the introduction of an unintentional mutation in clone 3A, the diminished activity of clone 3A demonstrates the importance of Trp68 in Zeocin binding.

In using focused libraries for directed evolution approaches, identification of multiple clones expressing variants containing identical mutations is typically one indication that the libraries have been screened exhaustively. In the present study, multiple clones were identified with identical sequences on few occasions, indicating additional beneficial mutations of BRP are likely to be identified through further screening of the libraries.

Minimal variation in Zeocin sensitivity due to BRP copy number or due to extreme variability in protein expression levels was expected because the transformants all express the She ble gene (BRP) integrated at precisely the same genomic site. Nonetheless, based on previous experience with antibody libraries expressed in bacteria, it is possible that single amino acid mutations affect the precise amount of BRP protein. Therefore, the expression levels of BRP protein in clones displaying altered sensitivities to Zeocin were assessed by Western blot and ELISA using a rabbit polyclonal antibody raised against BRP.

For quantitation of BRP variants by Western blotting, approximately equivalent amounts of total cell protein (as determined by the BCA protein assay) from different BRP clones were resolved by sodium dodecyl sulfate (SDS-PAGE) and transferred to nitrocellulose in two different experiments. Ponceau S staining of the blots for protein prior to probing with the BRP antibody revealed that near equivalent amounts of total protein from the various samples was loaded or used to assess relative protein expression.

Cell lysates from clones expressing beneficial, detrimental, and silent mutations, as well as wild type BRP were prepared. Equivalent quantities of total cell protein were resolved by SDS-PAGE, transferred to nitrocellulose, and probed with the rabbit antibody. The relative signal obtained from the clones, regardless of the mutation, was comparable and demonstrated that the expression levels were similar. In addition, equivalent quantities of total cell protein were incubated on a microtiter plate coated with the polyclonal rabbit anti-BRP antibody. ELISA quantitation of the BRP present in the various cell extracts following incubation with biotinylated rabbit anti-BRP antibody and streptavidin-alkaline phosphatase conjugate was consistent with the Western blot quantitation of BRP and demonstrated that the extracts contained similar quantities of BRP. The small differences in the relative expression levels of the BRP variants (less than 10-fold variation between samples) are very similar to the differences in antibody expression levels observed in bacterial systems (Watkins et al., Anal. Biochem. 253:37-45 (1997)). Thus, the differences in Zeocin sensitivity displayed by cells expressing BRP variants likely reflect the affinity of BRP for Zeocin and not differences in the relative amounts of BRP. Variants are purified to obtain precise measurement of their affinity constants.

These results demonstrate the expression and screening of a library of protein variants in mammalian cells. The variants can be screened for alterations in activity or function.

### EXAMPLE XI

### Expression of Butyrylcholinesterase Variant Libraries in Mammalian Cells

This example describes the expression of butyrylcholinesterase variant libraries in mammalian cells.

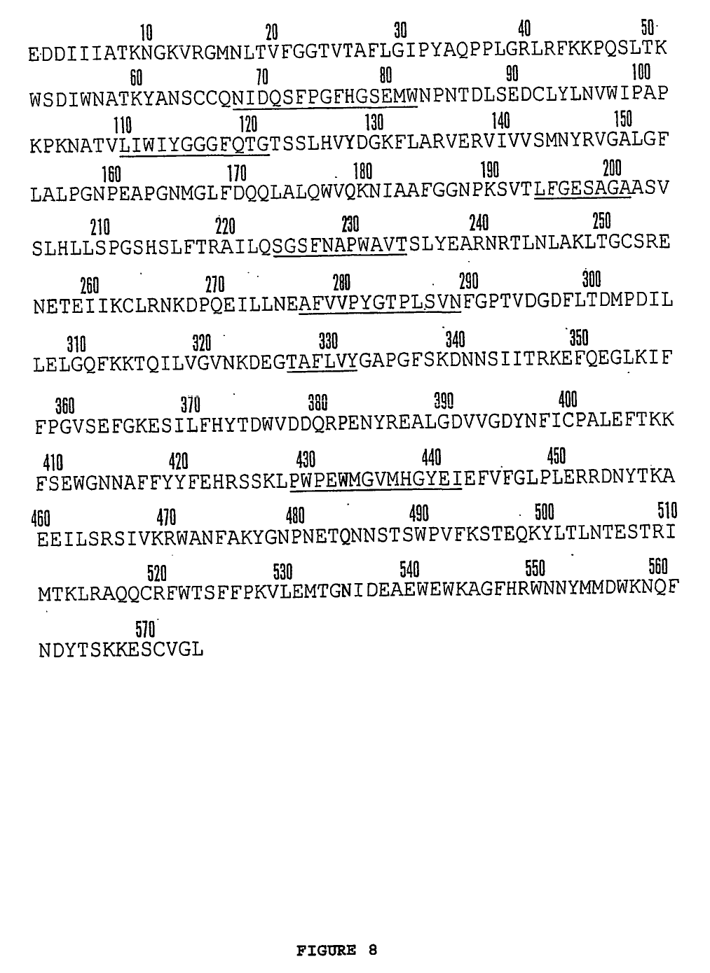

Studies with cholinesterases have revealed that the catalytic triad and other residues involved in ligand binding are positioned within a deep, narrow, active-site gorge rich in hydrophobic residues (reviewed in Soreq et al., Trends Biochem. Sci. 17:353-358 (1992)). The sites of seven focused libraries of butyrylcholinesterase variants (FIG. 8, underlined residues) were selected to include amino acids determined to be lining the active site gorge. The seven regions correspond to amino acids 68-82, 110-121, 194-201, 224-234, 277-289, 327-332, and 429-442 (see underlined sequences in FIG. 8).

The seven regions of butyrylcholinesterase selected for focused library synthesis span residues that include the 8 aromatic active site gorge residues (W82, W112, Y128, W231, F329, Y332, W430 and Y440) as well as two of the catalytic triad residues. The integrity of intrachain disulfide bonds, located between ⁶⁵Cys-⁹⁵Cys, ²⁵²Cys-²⁶³Cys, and ⁴⁰⁰Cys⁻⁵¹⁹Cys is maintained to ensure functional butyrylcholinesterase structure. In addition, putative glycosylation sites (N—X—S/T) located at residues 17, 57, 106, 241, 256, 341, 455, 481, 485, and 486 also are avoided in the library syntheses. In total, the seven focused libraries span 79 residues, representing approximately 14% of the butyrylcholinesterase linear sequence, and result in the expression of about 1500 distinct butyrylcholinesterase variants. Libraries of nucleic acids corresponding to the seven regions of human butyrylcholinesterase to be mutated are synthesized by codon-based mutagenesis (see U.S. Pat. Nos. 5,264,563 and 5,523,388; Glaser et al. J. Immunology 149:3903-3913 (1992)).

The oligonucleotides encoding the butyrylcholinesterase variants containing a single amino acid mutation is cloned into the doublelox targeting vector using oligonucleotide-directed mutagenesis (Kunkel, supra, 1985). To improve the mutagenesis efficiency and diminish the number of clones expressing wild-type butyrylcholinesterase, the libraries are synthesized in a two-step process. In the first step, the butyrylcholinesterase DNA sequence corresponding to each library site is deleted by hybridization mutagenesis. In the second step, uracil-containing single-stranded DNA for each deletion mutant, one deletion mutant corresponding to each library, is isolated and used as template for synthesis of the libraries by oligonucleotide-directed mutagenesis. This approach has been used routinely for the synthesis of antibody libraries and results in more uniform mutagenesis by removing annealing biases that potentially arise from the differing DNA sequence of the mutagenic oligonucleotides. In addition, the two-step process decreases the frequency of wild-type sequences relative to the variants in the libraries, and consequently makes library screening more efficient by eliminating repetitious screening of clones encoding wild-type butyrylcholinesterase.

The quality of the libraries and the efficiency of mutagenesis is characterized by obtaining DNA sequence from approximately 20 randomly selected clones from each library. The DNA sequences demonstrate that mutagenesis occurs at multiple positions within each library and that multiple amino acids were expressed at each position. Furthermore, DNA sequence of randomly selected clones demonstrates that the libraries contain diverse clones and are not dominated by a few clones.

As shown in Table 5, several cell lines and transfection methods were characterized for expression of butyrylcholinesterase variants. The cells tested for transfection were NIH3T3 (13-1) cells, Chinese hamster ovary (CHO) cells, and 293T human embryonic kidney cells. Both Flp recombinase and Cre recombinase were tested for stable transfection. Lipid-based transient transfection was also tested.

TABLE 5
Expression of a single butyrylcholinesterase
variant per cell using either stable or transient cell
transfection.

| Cell | | Integration | Integration? | Integration? |
|---|---|---|---|---|
| Line | Expression | Method | (PCR) | (Activity) |
| NIH3T3 | Transient | N/A | N/A | Transient, |
| (13-1) | (lipid- | | | very low |
| | based) | | | activity |
| NIH3T3 | Stable | Cre | Yes | No measurable |
| (13-1) | | recombinase | | activity |
| CHO | Transient | N/A | N/A | Transient, |
| | (lipid- | | | measurable |
| | based) | | | activity |
| | | | | (colorimetric |
| | | | | and cocaine |
| | | | | hydrolysis) |
| 293 | Transient | N/A | N/A | Transient, |
| | (lipid- | | | measurable |
| | based) | | | activity |
| | | | | (colorimetric |
| | | | | and cocaine |
| | | | | hydrolysis) |
| 293 | Stable | Flp | Yes | Measurable |
| | | recombinase | | activity |
| | | | | (colorimetric |
| | | | | and cocaine |
| | | | | hydrolysis) |

These results demonstrate the expression of a single butyrylcholinesterase variant per cell using either stable or transient cell transfection.

Each of the seven libraries of butyrylcholinesterase variants are transformed into a host mammalian cell line using the doublelox targeting vector and the optimized transfection conditions described in Example VIII. Following Cre-mediated transformation, the host cells are plated at limiting dilutions to isolate distinct clones in a 96-well format. Cells with the butyrylcholinesterase variants integrated in the Cre/lox targeting site are selected with geneticin. Subsequently, the DNA encoding butyrylcholinesterase variants from 20-30 randomly selected clones from each library are sequenced and analyzed as described above. Briefly, total cellular DNA is isolated from about 10⁴ cells of each clone of interest using DNeasy Tissue Kits (Qiagen; Valencia, Calif.). The butyrylcholinesterase gene is amplified using PfuTurbo DNA polymerase (Stratagene; La Jolla, Calif.), and an aliquot of the PCR product is then used for sequencing the DNA encoding butyrylcholinesterase variants from randomly selected clones by the fluorescent dideoxynucleotide termination method (Perkin-Elmer, Norwalk, Conn.) using a nested oligonucleotide primer. Sequencing demonstrates uniform introduction of the library, and the diversity of mammalian transformants resembles the diversity of the library in the doublelox targeting vector following transformation of bacteria.

A library corresponding to the region corresponding to amino acids 277-289 of butyrylcholinesterase was expressed, and individual variants were screened by measuring the hydrolysis of [³H]-cocaine using the microtiter assay. The catalytic efficiency (Vₘₐₓ/Kₘ) of variants with enhanced activity were characterized using the microtiter assay to determine their relative Kₘ and Vₘₐₓ. Briefly, butyrylcholinesterase from culture supernatants are immobilized using a capture reagent, such as an antibody, that is saturated at low butyrylcholinesterase concentrations as described previously by Watkins et al.,

Anal. Biochem. 253: 37-45 (1997). As a result, butyrylcholinesterase from dilute samples is concentrated and uniform quantities of different butyrylcholinesterase variant clones are immobilized, regardless of the initial concentration of butyrylcholinesterase in the culture supernatant. Subsequently, unbound butyrylcholinesterase and other culture supernatant components that potentially interfere with the assay, such as unrelated serum or cell-derived proteins with significant esterase activity, are washed away and the activity of the immobilized butyrylcholinesterase is determined. The assay is performed in a microtiter format using a commercially available rabbit anti-human cholinesterase polyclonal antibody (DAKO, Carpinteria, Calif.). Unbound material is removed by washing with 100 mM Tris, pH 7.4, and the amount of active butyrylcholinesterase captured is quantitated by measuring butyrylthiocholine hydrolysis or formation of benzoic acid. The assay can be performed with a radioactive benzoic acid tracer, in which the solubility difference at pH 3.0 between substrate (for example, cocaine, insoluble) and product (for example, benzoic acid, soluble) is exploited, or by HPLC (Xie et al., Mol. Pharmacol. 55:83-91 (1999)).

The kinetic constants for wild-type butyrylcholinesterase and the variants are determined and used to compare the catalytic efficiency of the variants relative to wild-type butyrylcholinesterase. Kₘ values for (−)-cocaine are determined at 37° C. Vₘₐₓ and Kₘ values are calculated using Sigma Plot (Jandel Scientific, San Rafael, Calif.). The number of active sites of butyrylcholinesterase is determined by the method of residual activity using echothiopate iodide or diisopropyl fluorophosphates as titrants, as described previously by Masson et al., Biochemistry 36: 2266-2277 (1997). Alternatively, the number of butyrylcholinesterase active sites is estimated using an ELISA to quantitate the mass of butyrylcholinesterase or butyrylcholinesterase variants present in culture supernatants. Purified human butyrylcholinesterase is used as the standard for the ELISA quantitation assay. The catalytic rate constant, k_{cat}, is calculated by dividing Vₘₐₓ by the concentration of active sites. Finally, the catalytic efficiencies of the variants are compared to wild-type butyrylcholinesterase by determining k_{cat}/Kₘ for each butyrylcholinesterase variant. In addition to the microtiter-based assay, the activity of the clones can be demonstrated in solution phase with product formation measured by the HPLC assay to verify the increased cocaine hydrolysis activity of the butyrylcholinesterase variants and confirm that the enhanced hydrolysis is at the benzoyl ester group.

Briefly, variant libraries corresponding to the region of butyrylcholinesterase corresponding to amino acids 277-289 of butyrylcholinesterase (FIG. 8) were transfected into mammalian cells, the 293T cell line, using Flp recombinase. Table 6 shows the butyrylcholinesterase variants S287G, P285Q and P285S that were identified and characterized utilizing Flp recombinase and the 293T human cell line. Three butyrylcholinesterase variants were identified that have enhanced cocaine hydrolase activity: S287G, P285Q and P285S (see Table 6).

TABLE 6
Identification and characterization of
butyrylcholinesterase variants with enhanced cocaine
hydrolase activity.

| Clone | Sequence | Relative Vₘₐₓ /Kₘ |
|---|---|---|
| 5.2.390F | Wild-type human BChE | 1.00 |
| | A328W | 13.4 |
| 5.2.258F | S287G | 4.3 |
| 5.2.444F | P285Q | 3.9 |
| 5.2.600F | P285S | 2.8 |

To generate combinatorial butyrylcholinesterase variant libraries, the beneficial mutations identified from screening libraries of butyrylcholinesterase variants containing a single amino acid mutation are combined in vitro to further improve the butyrylcholinesterase cocaine hydrolysis activity. The best mutations identified from screening the seven focused butyrylcholinesterase libraries are used to synthesize a combinatorial library. The combinatorial library is synthesized by oligonucleotide-directed mutagenesis, characterized, and expressed in the mammalian host cell line. Variants are screened and characterized as described above. DNA sequencing is used to reveal additive mutations.

This example demonstrates that butyrylcholinesterase variants can be generated and expressed in mammalian cells using a recombinase system and screened for enhanced activity.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A cell composition comprising a population of non-yeast eukaryotic cells containing a diverse population of about 10 or more variant nucleic acids, each of said variant nucleic acids being expressed in a different cell and located within each cell at an identical site in the genome.

2. The cell composition of claim 1, wherein said variant nucleic acids have predetermined amino acid changes at preselected positions within a parent amino acid sequence.

3. The cell composition of claim 1, wherein said variant nucleic acids are integrated in each cell by a site specific recombination sequence.

4. The cell composition of claim 1, wherein said cells express Cre recombinase or Flp recombinase.

5. The cell composition of claim 1, wherein said site in the genome comprises two lox sites.

6. The cell composition of claim 5, wherein at least one of said lox sites is a loxP site.

7. The cell composition of claim 5, wherein at least one of said lox sites is a lox511 site.

8. The cell composition of claim 5, wherein said site in the genome comprises two non-identical lox sites.

9. The cell composition of claim 8, wherein said site in the genome comprises a loxP site and a lox511 site.

10. The cell composition of claim 1, wherein said cell is a mammalian cell.

11. A method of identifying a polypeptide exhibiting optimized activity, comprising:
(a) screening the cell composition of claim 1 for an activity associated with a parent polypeptide of a diverse population of variant polypeptides encoded by said variant nucleic acids; and
(b) identifying a variant polypeptide exhibiting an optimized activity relative to said parent polypeptide.

12. A method of identifying a binding ligand, comprising:
(a) contacting the cell composition of claim 1 with one or more ligands; and
(b) identifying a ligand that binds to one of said variant nucleic acids.

13. A method of identifying a binding ligand, comprising:
(a) contacting the cell composition of claim 1 with one or more ligands, said cells containing a diverse population of variant polypeptides encoded by said variant nucleic acids; and
(b) identifying a ligand that binds to a polypeptide encoded by said variant nucleic acids.

14. A cell composition comprising a population of non-yeast eukaryotic cells containing a population of 10 or more variant nucleic acids, each of said variant nucleic acids being expressed in a different cell and integrated in the genome of each cell by a site specific recombination sequence.

15. The cell composition of claim 14, wherein said variant nucleic acids have predetermined amino acid changes at preselected positions within a parent amino acid sequence.

16. The cell composition of claim 14, wherein said cells express Cre recombinase or Flp recombinase.

17. The cell composition of claim 14, wherein said site in the genome comprises two lox sites.

18. The cell composition of claim 17, wherein at least one of said lox sites is a loxP site.

19. The cell composition of claim 17, wherein at least one of said lox sites is a lox511 site.

20. The cell composition of claim 17, wherein said site in the genome comprises two non-identical lox sites.

21. The cell composition of claim 20, wherein said site in the genome comprises a loxP site and a lox511 site.

22. The cell composition of claim 14, wherein said variant nucleic acids are integrated at a single site in the genome of each cell.

23. The cell composition of claim 14, wherein each of said variant nucleic acids is expressed in a different cell.

24. The cell composition of claim 14, wherein said cell is a mammalian cell.

25. A method of identifying a polypeptide exhibiting optimized activity, comprising:
(a) screening the cell composition of claim 14 for an activity associated with a parent polypeptide of a diverse population of variant polypeptides encoded by said variant nucleic acids; and
(b) identifying a variant polypeptide exhibiting an optimized activity relative to said parent polypeptide.

26. A method of identifying a binding ligand, comprising:
(a) contacting the cell composition of claim 14 with one or more ligands; and
(b) identifying a ligand that binds to one of said variant nucleic acids.

27. A method of identifying a binding ligand, comprising:
(a) contacting the cell composition or of claim 14 with one or more ligands, said cells containing a diverse population of variant polypeptides encoded by said variant nucleic acids; and
(b) identifying a ligand that binds to a polypeptide encoded by said variant nucleic acids.

28. A cell composition comprising a population of non-yeast eukaryotic cells containing a diverse population of 10 or more heterologous nucleic acid fragments, said heterologous nucleic acid fragments comprising distinct species of nucleic acid fragments, each of said heterologous nucleic acid fragments being expressed in a different cell and located within each cell at an identical site in the genome.

29. The cell composition of claim 28, wherein said heterologous nucleic acid fragments are integrated in each cell by a site specific recombination sequence.

30. The cell composition of claim 28, wherein said cells express Cre recombinase or Flp recombinase.

31. The cell composition of claim 28, wherein said site in the genome comprises two lox sites.

32. The cell composition of claim 31, wherein at least one of said lox sites is a loxP site.

33. The cell composition of claim 31, wherein at least one of said lox sites is a lox511 site.

34. The cell composition of claim 31, wherein said site in the genome comprises two non-identical lox sites.

35. The cell composition of claim 34, wherein said site in the genome comprises a loxP site and a lox511 site.

36. The cell composition of claim 28, wherein said cell is a mammalian cell.

37. A method of identifying a binding ligand, comprising:
(a) contacting the cell composition of claim 28 with one or more ligands; and
(b) identifying a ligand that binds to one of said heterologous nucleic acid fragments.

38. A method of identifying a binding ligand, comprising:
(a) contacting the cell composition of claim 28 with one or more ligands, said cells containing a diverse population of polypeptides encoded by said heterologous nucleic acid fragments; and
(b) identifying a ligand that binds to a polypeptide encoded by said heterologous nucleic acid fragments.

39. A method of identifying a polypeptide receptor for a ligand, comprising:
(a) contacting a population of non-yeast eukaryotic cells containing a diverse population of 10 or more heterologous nucleic acid fragments encoding polypeptides with a ligand, said heterologous nucleic acid fragments comprising distinct species of nucleic acid fragments, each of said heterologous nucleic acid fragments being expressed in a different cell and located within each cell at an identical site in the genome; and
(b) identifying a polypeptide encoded by said heterologous nucleic acid fragments that binds to said ligand.

40. A method of identifying a functional polypeptide fragment, comprising:
(a) introducing a diverse population of 10 or more heterologous nucleic acid fragments into a non-yeast eukaryotic cell to generate a population of cells, said heterologous nucleic acid fragments comprising distinct species of nucleic acid fragments, each of said nucleic acid fragments being expressed in a different cell and located within each cell at an identical site in the genome;
(b) screening said population of cells for a functional activity; and
(c) identifying a polypeptide encoded by said nucleic acid fragments having said functional activity.


SEQUENCE LISTING (90 sequences)

SEQ ID NO: 1 - DNA, 24 bp, Mus musculus
  CDS (1)...(24)
agctcaagtgtaagtttcatgaac

SEQ ID NO: 2 - protein, 8 aa, Mus musculus
SSSVSFMN

SEQ ID NO: 3 - DNA, 24 bp, Artificial Sequence
  synthetic variant
agctcaagtgtaaggttcatgaac

SEQ ID NO: 4 - protein, 8 aa, Artificial Sequence
  synthetic variant
SSSVRFMN

SEQ ID NO: 5 - DNA, 24 bp, Artificial Sequence
  synthetic variant
agcgagagtgtaaatcttatgaac

SEQ ID NO: 6 - protein, 8 aa, Artificial Sequence
  synthetic variant
SESVNLMN

SEQ ID NO: 7 - DNA, 24 bp, Artificial Sequence
  synthetic variant
agctcaagtgttaatttcatgaac

SEQ ID NO: 8 - protein, 8 aa, Artificial Sequence
  synthetic variant
SSSVNFMN

SEQ ID NO: 9 - DNA, 24 bp, Artificial Sequence
  synthetic variant
agctcaacggtaagtttcatgaac

SEQ ID NO: 10 - protein, 8 aa, Artificial Sequence
  synthetic variant
SSTVSFMN

SEQ ID NO: 11 - DNA, 24 bp, Artificial Sequence
  synthetic variant
agctcaagtgtagcgtatatgaac

SEQ ID NO: 12 - protein, 8 aa, Artificial Sequence
  synthetic variant
SSSVAYMN

SEQ ID NO: 13 - DNA, 24 bp, Artificial Sequence
  synthetic variant
agccagagtgctaagcatatgaac

SEQ ID NO: 14 - protein, 8 aa, Artificial Sequence
  synthetic variant
SQSAKHMN

SEQ ID NO: 15 - DNA, 24 bp, Artificial Sequence
  synthetic variant
gccacatccaatttggcttctgga

SEQ ID NO: 16 - protein, 8 aa, Artificial Sequence
  synthetic variant
ATSNLASG

SEQ ID NO: 17 - DNA, 24 bp, Artificial Sequence
  synthetic variant
gccacagagaagttggcttctgga

SEQ ID NO: 18 - protein, 8 aa, Artificial Sequence
  synthetic variant
ATEKLASG

SEQ ID NO: 19 - DNA, 24 bp, Artificial Sequence
  synthetic variant
gccacagttaatttggcttctgga

SEQ ID NO: 20 - protein, 8 aa, Artificial Sequence
  synthetic variant
ATVNLASG

SEQ ID NO: 21 - DNA, 24 bp, Artificial Sequence
  synthetic variant
gccacagtgaatttggcttctgga

SEQ ID NO: 22 - protein, 8 aa, Artificial Sequence
  synthetic variant
ATVNLASG

SEQ ID NO: 23 - DNA, 24 bp, Artificial Sequence
  synthetic variant
gccacatccagggcggcttctgga

SEQ ID NO: 24 - protein, 8 aa, Artificial Sequence
  synthetic variant
ATSRAASG

SEQ ID NO: 25 - DNA, 24 bp, Artificial Sequence
  synthetic variant
gccacacagaatttggcttctgga

SEQ ID NO: 26 - protein, 8 aa, Artificial Sequence
  synthetic variant
ATQNLASG

SEQ ID NO: 27 - DNA, 24 bp, Artificial Sequence
  synthetic variant
gccacatccaatttggcttctgga

SEQ ID NO: 28 - protein, 8 aa, Artificial Sequence
  synthetic variant
ATSNLASG

SEQ ID NO: 29 - DNA, 34 bp, bacteriophage P1
ataacttcgtataatgtatgctatacgaagttat

SEQ ID NO: 30 - DNA, 34 bp, Artificial Sequence
  mutant lox P
ataacttcgtataatgtatactatacgaagttat

SEQ ID NO: 31 - protein, 124 aa, Streptoalloteichus hindustanus
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 32 - protein, 134 aa, Staphylococcus aureus plasmid pUB110
MRMLQSIPALPVGDIKKSIGFYCDKLGFTLVHHEDGFAVLMCNEVRIHLWEASDEGWRSRSNDSPVCTGAESFIAGTASCRIEVEGIDELYQHIKPLGILHPNTSLKDQWWDERDFAVIDPDNNLISFFQQIKS

SEQ ID NO: 33 - protein, 126 aa, E. coli transposon Tn5
MTDQATPNLPSRDFDSTAAFYERLGFGIVFRDAGWMILQRGDLMLEFFAHPGLDPLASWFSCCLRLDDLAEFYRQCKSVGIQETSSGYPRIHAPELQEWGGTMAALVDPDGTLLRLIQNELLAGIS

SEQ ID NO: 34 - protein, 8 aa, Artificial Sequence
  BRP variant
DFVEDDFA

SEQ ID NO: 35 - protein, 8 aa, Artificial Sequence
  BRP variant
RFVEDDFA

SEQ ID NO: 36 - protein, 8 aa, Artificial Sequence
  BRP variant
DLVEDDFA

SEQ ID NO: 37 - protein, 8 aa, Artificial Sequence
  BRP variant
DSVEDDFA

SEQ ID NO: 38 - protein, 8 aa, Artificial Sequence
  BRP variant
DGVEDDFA

SEQ ID NO: 39 - protein, 8 aa, Artificial Sequence
  BRP variant
DFCEDDFA

SEQ ID NO: 40 - protein, 8 aa, Artificial Sequence
  BRP variant
DFVYDDFA

SEQ ID NO: 41 - protein, 8 aa, Artificial Sequence
  BRP variant
DFVELDFA

SEQ ID NO: 42 - protein, 8 aa, Artificial Sequence
  BRP variant
DFVEGDFA

SEQ ID NO: 43 - protein, 8 aa, Artificial Sequence
  BRP variant
DFVEDDSA

SEQ ID NO: 44 - protein, 8 aa, Artificial Sequence
  BRP variant
DFVEDDFR

SEQ ID NO: 45 - protein, 10 aa, Artificial Sequence
  BRP variant
VTLFISAVQD

SEQ ID NO: 46 - protein, 10 aa, Artificial Sequence
  BRP variant
LTLFISAVQD

SEQ ID NO: 47 - protein, 10 aa, Artificial Sequence
  BRP variant
ATLFISAVQD

SEQ ID NO: 48 - protein, 10 aa, Artificial Sequence
  BRP variant
VTLLISAVQD

SEQ ID NO: 49 - protein, 10 aa, Artificial Sequence
  BRP variant
VTLFVSAVQD

SEQ ID NO: 50 - protein, 10 aa, Artificial Sequence
  BRP variant
VTLFINAVQD

SEQ ID NO: 51 - protein, 10 aa, Artificial Sequence
  BRP variant
VTLFIIAVQD

SEQ ID NO: 52 - protein, 10 aa, Artificial Sequence
  BRP variant
VTLFISAVTD

SEQ ID NO: 53 - protein, 10 aa, Artificial Sequence
  BRP variant
VTLFISAVHD

SEQ ID NO: 54 - protein, 10 aa, Artificial Sequence
  BRP variant
VTLFISAVQP

SEQ ID NO: 55 - protein, 9 aa, Artificial Sequence
  BRP variant
DNTLAWVWV

SEQ ID NO: 56 - protein, 9 aa, Artificial Sequence
  BRP variant
DDTLGWVWV

SEQ ID NO: 57 - protein, 9 aa, Artificial Sequence
  BRP variant
DLTLGWVWV

SEQ ID NO: 58 - protein, 9 aa, Artificial Sequence
  BRP variant
DNPLGWVWV

SEQ ID NO: 59 - protein, 9 aa, Artificial Sequence
  BRP variant
DNTMGWVWV

SEQ ID NO: 60 - protein, 9 aa, Artificial Sequence
  BRP variant
DNTLCWVWV

SEQ ID NO: 61 - protein, 9 aa, Artificial Sequence
  BRP variant
DNTLSWVWV

SEQ ID NO: 62 - protein, 9 aa, Artificial Sequence
  BRP variant
DNTLGWWWV

SEQ ID NO: 63 - protein, 9 aa, Artificial Sequence
  BRP variant
DNTLGWVRV

SEQ ID NO: 64 - protein, 9 aa, Artificial Sequence
  BRP variant
DNTLGWVWL

SEQ ID NO: 65 - protein, 9 aa, Artificial Sequence
  BRP variant
DNTLAWVWC

SEQ ID NO: 66 - protein, 13 aa, Artificial Sequence
  BRP variant
TEIGEQPWGREFA

SEQ ID NO: 67 - protein, 13 aa, Artificial Sequence
  BRP variant
VEIGEQPWGREFA

SEQ ID NO: 68 - protein, 13 aa, Artificial Sequence
  BRP variant
TSIGEQPWGREFA

SEQ ID NO: 69 - protein, 13 aa, Artificial Sequence
  BRP variant
TEIGWQPWGREFA

SEQ ID NO: 70 - protein, 13 aa, Artificial Sequence
  BRP variant
TEIGEHPWGREFA

SEQ ID NO: 71 - protein, 13 aa, Artificial Sequence
  BRP variant
TEIGEQPLGREFA

SEQ ID NO: 72 - protein, 13 aa, Artificial Sequence
  BRP variant
TEIGEQPWGREGA

SEQ ID NO: 73 - protein, 13 aa, Artificial Sequence
  BRP variant
TEIGEQPWGREFS

SEQ ID NO: 74 - protein, 8 aa, Artificial Sequence
  BRP variant
DFYEDDFA

SEQ ID NO: 75 - protein, 8 aa, Artificial Sequence
  BRP variant
DFVEDNFA

SEQ ID NO: 76 - protein, 8 aa, Artificial Sequence
  BRP variant
FFVEDDFA

SEQ ID NO: 77 - protein, 8 aa, Artificial Sequence
  BRP variant
CFVEDDFA

SEQ ID NO: 78 - protein, 8 aa, Artificial Sequence
  BRP variant
GFVEDDFA

SEQ ID NO: 79 - protein, 10 aa, Artificial Sequence
  BRP variant
VILFISAVQD

SEQ ID NO: 80 - protein, 10 aa, Artificial Sequence
  BRP variant
VTLFISTVQD

SEQ ID NO: 81 - protein, 10 aa, Artificial Sequence
  BRP variant
VTLFISALQD

SEQ ID NO: 82 - protein, 9 aa, Artificial Sequence
  BRP variant
DNTLAWVLV

SEQ ID NO: 83 - protein, 9 aa, Artificial Sequence
  BRP variant
DNTSGWVWV

SEQ ID NO: 84 - protein, 9 aa, Artificial Sequence
  BRP variant
DNTLGWVLV

SEQ ID NO: 85 - protein, 9 aa, Artificial Sequence
  BRP variant
DNTLGWVCV

SEQ ID NO: 86 - protein, 13 aa, Artificial Sequence
  BRP variant
TPIGEQPWGREFA

SEQ ID NO: 87 - protein, 13 aa, Artificial Sequence
  BRP variant
TELGEQPWGREFA

SEQ ID NO: 88 - protein, 13 aa, Artificial Sequence
  BRP variant
TEIGSQPWGREFA

SEQ ID NO: 89 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 90 - DNA, 34 bp, Sacharomyces cervisiae
gaagttcctattctctagaaagtataggaacttc